(19) United States
(12) Patent Application Publication
(10) Pub. No.: US 2025/0382582 A1
(43) Pub. Date: Dec. 18, 2025

(54) SYSTEMS FOR STEM CELL PROGRAMMING AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US)

(21) Appl. No.: 19/024,149

(22) Filed: Jan. 16, 2025

Related U.S. Application Data
(63) Continuation of application No. PCT/US23/28033, filed on Jul. 18, 2023.
(60) Provisional application No. 63/390,474, filed on Jul. 19, 2022.

(51) Int. Cl. C12N 5/074; A61K 35/545; C12N 5/10; C12N 9/22

(52) U.S. Cl. CPC C12N 5/0696; A61K 35/545; C12N 5/10; C12N 9/22; C12N 2501/73; C12N 2506/1307; C12N 2510/00

(57) ABSTRACT

Provided herein are systems of modulating gene expression and methods of use thereof for converting cells of one type to another type (e.g., reprogramming of differentiated cells into stem cells).

### CROSS REFERENCE

This application is a continuation of International Patent Application No. PCT/US23/28033, filed Jul. 18, 2023, which claims the benefit of U.S. Provisional Patent Application No. 63/390,474, filed on Jul. 19, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 16, 2025, is named 61684-708-301_SL.xml and is 56,143 bytes in size.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell (e.g., a stem cell). In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species.

### SUMMARY

The present disclosure provide methods and systems for regulating expression or activity of target genes. Some aspects of the present disclosure provides methods and systems for differentiating and de-differentiating terminally differentiated cells. Some aspects of the present disclosure provides methods and systems for differentiating and de-differentiating stem cells.

In an aspect, the present disclosure provides a method for conversion of a plurality of cells of a first cell type (first plurality of cells) into a plurality of cells of a second cell type (second plurality of cells), the method comprising: contacting the first plurality of cells with a heterologous gene modulator exhibiting specific binding to a gene encoding HERV, to regulate an expression level or an epigenetic profile of HERV and effect the conversion from the first plurality of cells to the second plurality of cells.

In another aspect, the present disclosure provides a system for conversion of a plurality of cells of a first cell type (first plurality of cells) into a plurality of cells of a second cell type (second plurality of cells), the system comprising: a heterologous gene modulator exhibiting specific binding to a gene encoding HERV, to regulate an expression level or an epigenetic profile of the HERV and effect the conversion from the first plurality of cells to the second plurality of cells.

In another aspect, the present disclosure provides a method for conversion of a plurality of differentiated cells into a plurality of stem cells, the method comprising: contacting the plurality of differentiated cells with a heterologous genetic circuit comprising a plurality of gate units, wherein the heterologous genetic circuit is activatable to induce the plurality of gate units to modulate expression level or epigenetic profile of a plurality of distinct target endogenous genes in a sequential manner to effect the conversion, and wherein the plurality of gate units comprises: (i) a first gate unit that is preconfigured to regulate expression level or epigenetic profile of a first target endogenous gene of the plurality of distinct target endogenous genes, wherein the first target endogenous gene comprises embryo genome activation (EGA)-enriched Alu-motif (EEA); and ii) a second gate unit that is preconfigured to regulate expression level or epigenetic profile of a second target endogenous gene of the plurality of distinct target endogenous genes, wherein the second target endogenous gene comprises a cell de-differentiation factor selected from the group consisting of OCT4, SOX2, KLF4, and MYC, wherein, upon activation of the heterologous genetic circuit, the plurality of gate units operates to effect the conversion.

In another aspect, the present disclosure provides a system for conversion of a plurality of differentiated cells into a plurality of stem cells, the system comprising: a heterologous genetic circuit comprising a plurality of gate units, wherein the heterologous genetic circuit is activatable to induce the plurality of gate units to modulate expression level or epigenetic profile of a plurality of distinct target endogenous genes in a sequential manner to effect the conversion, wherein the plurality of gate units comprises: (i) a first gate unit that is preconfigured to regulate expression level or epigenetic profile of a first target endogenous gene of the plurality of distinct target endogenous genes, wherein the first target endogenous gene comprises embryo genome activation (EGA)-enriched Alu-motif (EEA); and (ii) a second gate unit that is preconfigured to regulate expression level or epigenetic profile of a second target endogenous gene of the plurality of distinct target endogenous genes, wherein the second target endogenous gene comprises a cell de-differentiation factor selected from the group consisting of OCT4, SOX2, KLF4, and MYC, wherein, upon activation of the heterologous genetic circuit, the plurality of gate units operates to effect the conversion.

In another aspect, the present disclosure provides a method for conversion of a plurality of differentiated cells into a plurality of stem cells, the method comprising: contacting the plurality of differentiated cells with a heterologous genetic circuit comprising a plurality of gate units, wherein the heterologous genetic circuit is activatable to induce the plurality of gate units to modulate expression level or epigenetic profile levels of a plurality of distinct target endogenous genes in a sequential manner to effect the conversion, and wherein the plurality of gate units comprises: (i) a first gate unit that is preconfigured to regulate expression level or epigenetic profile level of a first target endogenous gene of the plurality of distinct target endogenous genes, wherein the first target endogenous gene comprises a cell de-differentiation factor selected from the group consisting of OCT4, SOX2, KLF4, and MYC; and (ii) a second gate unit that is preconfigured to regulate expression level or epigenetic profile level of a second target endogenous gene of the plurality of distinct target endogenous genes, wherein the second target endogenous gene comprises a different cell de-differentiation factor selected from the group consisting of OCT4, SOX2, KLF4, and MYC, wherein, upon activation of the heterologous genetic circuit, the plurality of gate units operates to effect the conversion.

In another aspect, the present disclosure provides a system for conversion of a plurality of differentiated cells into a plurality of stem cells, the system comprising: a heterologous genetic circuit comprising a plurality of gate units, wherein the heterologous genetic circuit is activatable to induce the plurality of gate units to modulate expression level or epigenetic profile levels of a plurality of distinct target endogenous genes in a sequential manner to effect the conversion, and wherein the plurality of gate units comprises: (i) a first gate unit that is preconfigured to regulate expression level or epigenetic profile level of a first target endogenous gene of the plurality of distinct target endogenous genes, wherein the first target endogenous gene comprises a cell de-differentiation factor selected from the group consisting of OCT4, SOX2, KLF4, and MYC; and (ii) a second gate unit that is preconfigured to regulate expression level or epigenetic profile level of a second target endogenous gene of the plurality of distinct target endogenous genes, wherein the second target endogenous gene comprises a different cell de-differentiation factor selected from the group consisting of OCT4, SOX2, KLF4, and MYC, wherein, upon activation of the heterologous genetic circuit, the plurality of gate units operates to effect the conversion.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example of a heterologous genetic circuit.

FIG. 2 schematically illustrates a network of genes involved in cell pluripotency regulation.

FIG. 3 provides examples of heterologous genetic circuits, each showing stepwise modulation of target genes.

FIG. 4 shows fluorescent imaging of fibroblasts transfected with various conditions, each condition comprising a plasmid encoding a fluorescent protein and one or more plasmids required for each heterologous genetic circuit tested.

FIG. 5 shows fluorescent imaging of fibroblasts transfected with a plasmid encoding a fluorescent protein and a plasmids required for a heterologous genetic circuit for sequentially activating a plurality of target genes including embryo genome activation (EGA)-enriched Alu-motif (EEA) (top) and phase contrast imaging, Nanog-positive staining imaging, DNA-positive staining imaging, and Oct4-positive staining imaging to identify induced pluripotent cell reprogramming (bottom).

FIG. 6 shows phase contrast imaging of primary fibroblasts engineered with a heterologous genetic circuit for targeting human endogenous retrovirus (HERV) and EEA, to assess iPSC reprogramming and colony formation.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

### Definitions

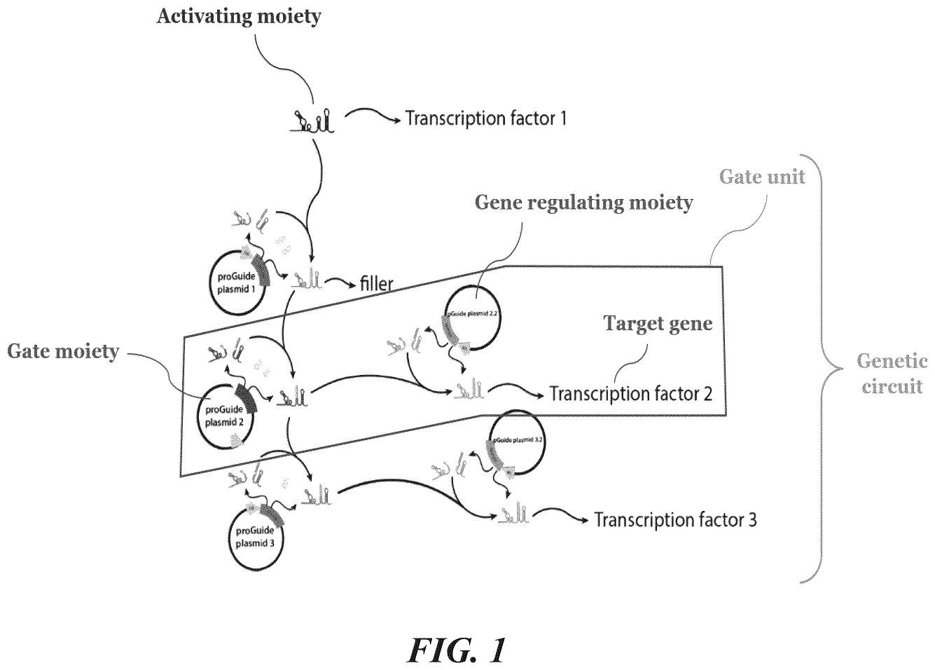

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 1).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units is activatable by an activating moiety (e.g., a heterologous input to the cell) to activate other gate units of the plurality of gate units (e.g., simultaneously at once, sequentially in a cascading manner, etc.) (FIG. 1). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 1). The terms “heterologous genetic circuit,” “HGC,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription/translation regulatory region, or series of regions, which the genetic switch acts on. The input for a gate unit can be an activating moiety and/or another gate unit. The output for a gate unit can be used to activate another gate unit, to de-activate another gate unit, to affect a target gene, and/or a combination of any of the above. For example, a gate unit can be comprised of a plurality of gate moieties and/or a plurality of gene regulating moieties (FIG. 1).

The term “activating moiety,” as referred to herein, generally refers to a moiety that can activate plurality of genetic circuits and/or a plurality of gate units. An activating moiety can be a heterologous input to a cell. In some cases, activating moieties can include, but are not limited to, a guide nucleic acid molecule (e.g., a gRNA) or other nucleic acid, polypeptides, polynucleotides, small molecules, light, or a combination thereof. For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties.

The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively or in addition to, a gate moiety can activate and/or deactivate another gate unit of the genetic circuit (FIG. 1). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 1). For example, a gene editing moiety can regulate expression of a gene by editing a nucleic acid sequence (e.g. CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g. CRISPR-Cas-inspired RNA targeting systems). Alternatively, a gene editing moiety can repress translation of a gene (e.g. Cas13).

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g. Cas12).

In some cases, a plasmid can encode a non-functional form of a gene editing moiety. The plasmid can be activated (e.g., genetically modified) to express a functional form of the gene editing moiety, e.g., via activation of a functional gate moiety. For example, the plasmid can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited (e.g., cleaved at one or more sites, then repaired via endogenous mechanisms (e.g., homologous recombination, nonhomologous end joining) to allow expression of a functional form of the gene editing moiety (e.g., a functional form of the guide nucleic acid molecule with specific binding to the target gene of the cell), to permit modulation of the target gene in the cell.

In some cases, a gene regulating moiety can comprise a nucleic acid molecule (e.g., a guide nucleic acid molecule that forms a complex with an endonuclease, such as a Cas protein). Alternatively or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

The abovementioned Cas proteins can form a complex with a guide nucleic acid (gNA (e.g., a guide RNA (gRNA)) and utilize the gNA to specifically bind to a target polynucleotide sequence (e.g., a target DNA sequence, a target RNA sequence). Accordingly, in some cases, such Cas proteins may be referred to as a “NA-guided nuclease” (e.g., RNA-guided nuclease). As used herein, the term “guide nucleic acid” (gNA) can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence” or “spacer sequence”. A nucleic acid-targeting segment may comprise a sub-segment that may be referred to as a “protein binding segment” or “protein binding sequence” or “Cas protein binding segment” or “scaffold sequence.”

A gene regulating moiety can be a transcriptional modulator system (e.g., a gene repressor complex or a gene activator complex). For example, a gene regulating moiety can be a gene repressor complex comprising a dCas protein operatively coupled to (e.g., coupled to or fused with) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASC1, JMJD2D, JARID1A, RBP2, JARID1B/PLU-1, JARIDIC/SMCX, JARIDID/SMCY, HDACl, HDAC2, HDAC3, HDAC8, HDAC4, HDAC5, HDAC7, HDAC9, SIRT1, SIRT2, HDACl1, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SET1A, SET1B, MLL1, MLL2, MLL3, MLL4, MLL5, ASH1, SYMD2, NSD1, JHDM2a, JHDM2b, UTX, JMJD3, GCN5, PCAF, CBP, p300, TAF1, TIP60/PLIP, MOZ/MYST3, MORF/MYST4, SRCl, ACTR, P160, CLOCK, TET1CD, TET1, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TET1CD), TET1, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “sequence identity” generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 95%, or at least 98% sequence identity with any sequence provided herein.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [aS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “reprogramming,” “dedifferentiation,” “increasing cell potency,” or “increasing developmental potency,” as used interchangeable herein, generally refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “pluripotent” generally refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (i.e., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., natural killer (NK) cells). In some cases, iPSCs can be engineered to differentiate first into tissue-specific stem cells (e.g., hematopoietic stem cells (HSCs) or hematopoietic progenitor cells), which can be further induced to differentiate into committed cells (e.g., NK cells).

### Overview

Biological programming, such as cellular programming (e.g., the creation of iPSCs), allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., endogenous DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control a cascade of a plurality of desired expression and/or activity profiles of a plurality of genes in the cell. To allow for better control of specific cellular outcomes, genetic circuits can be multiplexed to create positive feedback and/or negative feedback systems.

Although CRISPR/Cas systems can be used for gene editing, Cas is essentially a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this simple barcoding, often using exogenous fluorophores, doesn't allow for the multiplexed regulation of endogenous genes to effect cell differentiation.

The overexpression of Yamanaka factors, a group of protein transcription factors which play an important role in the creation of iPSCs, can be used to induce pluripotency. However, the method of overexpressing Yamanaka factors is inefficient, slow, and stochastic. Adding to these issues, epigenetic markers can remain from the original differentiated cells, which can exacerbate problems in using iPSCs made with this method.

For example, there remains an unmet need for an activatable, multiplexed CRISPR/Cas system and use of the same to edit a target polynucleotide (e.g., a genome of a cell, in particular a eukaryotic cell), using cascades of gRNAs to form genetic circuits in order to single-handedly affect gene regulation and, in turn, the de-programming of cells to create iPSCs efficiently and in a way that removes epigenetic markers to improve the viability and use of iPSCs.

Thus, various aspects of the present disclosure provides systems, compositions, and methods thereof for regulating a target gene (e.g., an endogenous target gene) that is (i) derived from a virus and (ii) integrated in a genome of a non-viral cell, e.g., to regulate fate of the non-viral cell (e.g., conversion of the non-viral cell from a first cell type to a second cell type, such as de-differentiation). Additional aspects of the present disclosure provides systems, compositions, and methods thereof for regulating other target genes (e.g., additional endogenous target genes) to regulate fate of a non-viral cell. In some embodiments, Systems, compositions, and methods as provided herein may not and need not utilize a heterologous gene encoding such target gene(s), and instead rely on regulating endogenous genes. In some embodiments, the present disclosure provides systems and methods for engineering a CRISPR/Cas9 system, which includes a Cas endonuclease and an array of cognate single guide RNAs (sgRNA or gRNA) that harbor inactivation sequences in a non-essential region and are activatable, to allow for the derivation of iPSCs from differentiated cells. The present disclosure also provides for an engineered cell that can contain any of the above-mentioned systems or that can be capable of performing any of the above-mentioned methods.

### Systems and Methods for Conversion of Cells of One Type to Cells of Another Type

Various aspects of the present disclosure provide systems for inducing a desired expression and/or activity level (or profile thereof) of one or more target genes in a cell. Various aspects of the present disclosure provide methods for inducing a desired expression and/or activity level (or profile thereof) of one or more target genes in a cell.

In an aspect, the present disclosure provides for a system that induces a desired expression and/or activity profile of a target gene (e.g., a target gene that is derived from a virus) in a cell. The system can comprise a heterologous gene modulator that exhibits specific binding to the target gene. In some cases, the heterologous gene modulator can be a part of a heterologous genetic circuit that is introduced to the cell to induce the desired expression. For example, the heterologous gene modulator can include an endonuclease and/or a polynucleotides sequence (e.g., a Cas protein, a guide nucleic acid molecule, and/or a combination thereof).

In some cases, the heterologous genetic circuit can comprise at least one gate unit (e.g., a single gate unit, or a plurality of gate units). The plurality of gate units can comprise at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s). The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, or at most about 2 gate unit(s). The plurality of gate units can be different (e.g., comprising different polynucleotide sequences).

Alternatively, the expression and/or activity profile of the target gene of the cell as disclosed herein can be regulated in absence of a heterologous genetic circuit. For example, a gene regulating moiety (e.g., a CRISPR/Cas protein and a guide nucleic acid molecule against the target gene) configured to bind the target gene can be introduced (e.g., transfected or transduced) to the cell, such that the introduction can be sufficient to regulate the expression and/or activity profile of the target gene without a need for any further regulation of the activity of the gene regulating moiety.

A heterologous genetic circuit as disclosed herein can operate with a single gate unit, such that activation of the single gate unit can regulate expression or activity level of a target gene (e.g., a target endogenous gene), such as a virus-derived gene in a mammalian genome (e.g., HERV). Alternatively, or in addition to, a heterologous genetic circuit as disclosed herein can operate with a single gate unit, such that activation of the single gate unit can regulate the epigenetic profile of a target gene (e.g., a target endogenous gene), such as a virus-derived gene in a mammalian genome (e.g., HERV).

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel paths), or a combination thereof, e.g., to sequentially regulate expression or activity of a plurality of target genes (e.g., endogenous target genes), respectively, such as the virus-derived gene and an additional target gene.

Without wishing to be bound by theory, regulating (e.g., modifying) the gene expression level and/or epigenetic profile of the virus-derived gene in the mammalian genome (e.g., HERV) can prime the cell for enhanced regulation of cell fate. In some cases, the regulation of the expression level and/or epigenetic profile of the virus-derived gene can effect opening of at least a portion of a genome of the cell and/or resetting of the at least the portion of the cell such that epigenetic markers (e.g., DNA histone markers) can be ubiquitously removed. Alternatively, the regulation of the expression level and/or epigenetic profile of the virus-derived gene can effect opening of at least a portion of a genome of the cell and/or resetting of the at least the portion of the cell such that epigenetic markers (e.g., DNA histone markers) can be selectively added to specific positions. Alternatively, the regulation of the expression level and/or epigenetic profile of the virus-derived gene can effect opening of at least a portion of a genome of the cell and/or resetting of the at least the portion of the cell such that epigenetic markers (e.g., DNA histone markers) can be ubiquitously added. Alternatively, the regulation of the expression level and/or epigenetic profile of the virus-derived gene can effect opening of at least a portion of a genome of the cell and/or resetting of the at least the portion of the cell such that epigenetic markers (e.g., DNA histone markers) can be selectively removed from specific positions.

In some cases, the regulation of the expression level and/or epigenetic profile of the virus-derived gene can effect opening of at least a portion of a genome of the cell and/or resetting of the at least the portion of the cell such that the process of differentiating cells can be specifically targeted depending on the targeted element. In some cases, the regulation of the expression level and/or epigenetic profile of the virus-derived gene can effect opening of at least a portion of a genome of the cell and/or resetting of the at least the portion of the cell such that the process of differentiating cells can be specifically targeted depending on the target cell lineage.

In some cases, the regulation of the expression level and/or epigenetic profile of the virus-derived gene can effect opening of at least a portion of a genome of the cell and/or resetting of the at least the portion of the cell such that the process of de-differentiating cells can be specifically targeted depending on the targeted element. In some cases, the regulation of the expression level and/or epigenetic profile of the virus-derived gene can effect opening of at least a portion of a genome of the cell and/or resetting of the at least the portion of the cell such that the process of de-differentiating cells can be specifically targeted depending on the initial cell lineage.

A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g. cell proliferation assays, metabolic activity assays, cell killing assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

The outcome in the cell can comprise regulation of a target gene. The regulation of the target gene can comprise a plurality of distinct modulations of the target gene. The plurality of gate units can each induce one of the plurality of distinct modulations of the target gene, such that a collection of the distinct modulation in concert yields a final expression and/or activity profile of the target gene. At least two distinct modulations of the plurality of distinct modulations can both increase an expression and/or activity level of the target gene. At least two distinct modulations of the plurality of distinct modulations can both decrease an expression and/or activity level of the target gene. Alternatively, a first distinct modulation of the plurality of distinct modulations can increase an expression and/or activity level of the target gene, while a second distinct modulation of the plurality of distinct modulations can decrease the expression and/or activity level of the target gene. In such case, the first distinct modulation can occur prior to the second distinct modulation, or vice versa. Alternatively, a distinct modulation (e.g., a first and/or second modulation) of the plurality of distinct modulations can maintain an expression and/or activity level of the target gene at the level of expression and/or activity level prior to the modulation.

The outcome in the cell can comprise regulation of the epigenetic profile of a target gene. The regulation of the epigenetic profile of the target gene can comprise a plurality of distinct modulations of the target gene. The plurality of gate units can each induce one of the plurality of distinct modulations to the epigenetic profile of the target gene, such that a collection of the distinct modulation in concert yields a final epigenetic profile of the target gene. At least two distinct modulations of the plurality of distinct modulations can both modify an epigenome of the target gene. The epigenome of a target gene can be modified through the regulation of methylation, acetylation, phosphorylation, ubiquitylation, sumoylation, ribosylation, or citrullination. In such case, the first distinct modulation can occur prior to the second distinct modulation, or vice versa. Alternatively, a distinct modulation (e.g., a first and/or second modulation) of the plurality of distinct modulations can maintain an epigenetic state of the target gene.

In some cases, each distinct modulation of the plurality of distinct modulations of the target gene, as disclosed herein, can be necessary but individually insufficient to effect the desired expression and/or activity profile of the target gene. Thus, the outcome in the cell (e.g., enhanced cell function, induced cell state, etc.) induced by the plurality of distinct modulations of the target gene may not be possible in absence of any one of the plurality of distinct modulations of the target gene. Alternatively, a degree or measure of the outcome in the cell induced by the plurality of distinct modulations of the target gene can be greater than a degree or measure of the outcome in a control cell that is induced by none, one or more, but not all of the plurality of distinct modulations of the target gene, and/or by all of the plurality of distinct modulations of the target genes occurring through a different sequential order of events.

In some cases, each distinct modulation of the plurality of distinct modulations of the target gene, as disclosed herein, can be necessary but individually insufficient to effect the desired epigenetic profile of the target gene. Thus, the outcome in the cell (e.g., enhanced cell function, induced cell state, etc.) induced by the plurality of distinct modulations of the target gene may not be possible in absence of any one of the plurality of distinct modulations of the target gene. Alternatively, a degree or measure of the outcome in the cell induced by the plurality of distinct modulations of the epigenome of the target gene can be greater than a degree or measure of the outcome in a control cell that is induced by none, one or more, but not all of the plurality of distinct modulations of the epigenome of the target gene, and/or by all of the plurality of distinct modulations of the epigenomes of the target genes occurring through a different sequential order of events.

A second gate unit can be activated by a first gate unit (e.g. directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.).

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively or in addition to, inactivation can be achieved through the use of a transcriptional modulator system (e.g. a transcriptional repressor). An endonuclease-transcriptional modulator system (e.g., a Cas-repressor) can be used to achieve polynucleotide cleavage (e.g. for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively or in addition to, the endonuclease-transcriptional modulator system (e.g., a Cas-repressor) can be used to modulate target gene expression.

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

In some cases, a first gate unit modulates a first target gene. Alternatively, or in addition to, a first gate unit can also modulate a second gate unit. The modulation of the second gate unit can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or after the modulation of the first gate unit, as ascertained by rt-qPCR, Western blotting, or other methods.

In some cases, the second gate unit can modulate a second target gene. The modulation of the second target gene can occur at least or up to about 1 millisecond, at least or up to about 2 milliseconds, at least or up to about 3 milliseconds, at least or up to about 4 milliseconds, at least or up to about 5 milliseconds, at least or up to about 6 milliseconds, at least or up to about 7 milliseconds, at least or up to about 8 milliseconds, at least or up to about 9 milliseconds, at least or up to about 10 milliseconds, at least or up to about 20 milliseconds, at least or up to about 30 milliseconds, at least or up to about 40 milliseconds, at least or up to about 50 milliseconds, at least or up to about 60 milliseconds, at least or up to about 70 milliseconds, at least or up to about 80 milliseconds, at least or up to about 90 milliseconds, at least or up to about 100 milliseconds, at least or up to about 200 milliseconds, at least or up to about 300 milliseconds, at least or up to about 400 milliseconds, at least or up to about 500 milliseconds, at least or up to about 600 milliseconds, at least or up to about 700 milliseconds, at least or up to about 800 milliseconds, at least or up to about 900 milliseconds, at least or up to about 1 second, at least or up to about 2 seconds, at least or up to about 3 seconds, at least or up to about 4 seconds, at least or up to about 5 seconds, at least or up to about 6 seconds, at least or up to about 7 seconds, at least or up to about 8 seconds, at least or up to about 9 seconds, at least or up to about 10 seconds, at least or up to about 15 seconds, at least or up to about 20 seconds, at least or up to about 30 seconds, at least or up to about 40 seconds, at least or up to about 50 seconds, at least or up to about 1 minute, at least or up to about 2 minutes, at least or up to about 3 minutes, at least or up to about 4 minutes, at least or up to about 5 minutes, at least or up to about 6 minutes, at least or up to about 7 minutes, at least or up to about 8 minutes, at least or up to about 9 minutes, at least or up to about 10 minutes, at least or up to about 20 minutes, at least or up to about 30 minutes, at least or up to about 40 minutes, at least or up to about 50 minutes, at least or up to about 1 hour, at least or up to about 2 hours, at least or up to about 3 hours, at least or up to about 4 hours, at least or up to about 5 hours, at least or up to about 6 hours, at least or up to about 7 hours, at least or up to about 8 hours, at least or up to about 9 hours, at least or up to about 10 hours, at least or up to about 12 hours, at least or up to about 16 hours, at least or up to about 20 hours, or at least or up to about 24 hours, or more after the modulation of the first target gene, as ascertained by rt-qPCR, Western blotting, or other methods.

In some cases, modification of a target gene by a gate unit can inactivate a gene. For example, modification of a gene can stop expression and/or activity level of a target gene. Alternatively, modification of a gene can decrease the expression and/or activity level of a target gene. In some cases, modification of a gene can increase the expression and/or activity level of a target gene. Alternatively, modification of a gene can maintain the expression and/or activity level of a target gene.

An expression and/or activity profile of a gene of interest (e.g. a differentiation marker) can be compared to a control gene (e.g., a house keeping gene such as GAPDH), relative expression levels of two or more genes of interest (e.g., a ratio of expression or activity level between a stem cell marker and a differentiation marker), relative average expression levels of a gene of interest compared to average expression levels of that same gene of interest in a cell type of interest, etc.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least about 1, at least about 2, at least about 3, at least about 4, at least about 5, at least about 6, at least about 7, at least about 8, at least about 9, at least about 10, or more single-stranded breaks. In some cases, inactivation of a gene can be caused by at most about 10, at most about 9, at most about 8, at most about 7, at most about 6, at most about 5, at most about 4, at most about 3, at most about 2, or at most about 1 single-stranded breaks.

In some cases, a gNA can have a size (e.g., including both spacer sequence and scaffold sequence) of at least or up to about 60 nucleotides, at least or up to about 70 nucleotides, at least or up to about 80 nucleotides, at least or up to about 85 nucleotides, at least or up to about 90 nucleotides, at least or up to about 95 nucleotides, at least or up to about 100 nucleotides, at least or up to about 105 nucleotides, at least or up to about 110 nucleotides, at least or up to about 120 nucleotides, at least or up to about 130 nucleotides, at least or up to about 140 nucleotides, at least or up to about 150 nucleotides, or at least or up to about 200 nucleotides.

In some cases, a scaffold sequence of a gNA can have a size of at least or up to about 30 nucleotides, at least or up to about 35 nucleotides, at least or up to about 40 nucleotides, at least or up to about 45 nucleotides, at least or up to about 50 nucleotides, at least or up to about 55 nucleotides, at least or up to about 60 nucleotides, at least or up to about 65 nucleotides, at least or up to about 70 nucleotides, at least or up to about 75 nucleotides, at least or up to about 80 nucleotides, at least or up to about 85 nucleotides, at least or up to about 90 nucleotides, at least or up to about 95 nucleotides, at least or up to about 100 nucleotides, at least or up to about 100 nucleotides, at least or up to about 120 nucleotides, at least or up to about 130 nucleotides, at least or up to about 140 nucleotides, or at least or up to about 150 nucleotides.

In some cases, a spacer sequence of a gNA can have a size of at least or up to about 10 nucleotides, at least or up to about 11, at least or up to about 12, at least or up to about 13, at least or up to about 14, at least or up to about 15, at least or up to about 16, at least or up to about 17, at least or up to about 18, at least or up to about 19, at least or up to about 20, at least or up to about 21, at least or up to about 22, at least or up to about 23, at least or up to about 24, at least or up to about 25, at least or up to about 26, at least or up to about 27, at least or up to about 28, at least or up to about 29, or at least or up to about 30 nucleotides.

In some cases, the systems and methods of the present disclosure can utilize a single endonuclease system (e.g., a Cas-repressor) to achieve both (i) polynucleotide cleavage (e.g. for activating/inactivating the gate moiety and/or the gene regulating moiety) and (ii) modulation of target gene expression. When using a single endonuclease-transcriptional modulator system, unique guide nucleic acid molecules (gNAs) of differing spacer sequence lengths can be used to determine whether the single endonuclease-transcriptional modulator system may (i) hybridize to the polynucleotide sequence to induce Cas-mediated nuclease activity of the polynucleotide sequence, or (ii) can hybridize to a target gene (e.g., genomic DNA) to modulate expression and/or activity level of the target gene via action of the transcriptional activator without mediating Cas nuclease activity, as desired by the individual heterologous genetic circuit. For example, use of gNAs of differing spacer sequence lengths that bind to different targets can allow for a second gate unit as provided herein to induce inactivation of a first gate unit that has been activated and/or induce a distinct modulation of a second target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g. a Cas or a Cas-transcriptional modulator fusion protein) at a target polynucleotide sequence. Alternatively or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g. a Cas-activator system, a Cas-repressor system), without cleavage of the target gene.

In some cases, modification of a polynucleotide sequence (e.g., as a component of a gate unit, such as a gate moiety) or a target gene can be caused by a double-stranded break wherein there is a discontinuity in both nucleotide strands. In some cases, a number of such double-stranded break (e.g., necessary for such modification) can be at least or up to about 1, at least or up to about 2, at least or up to about 3, at least or up to about 4, at least or up to about 5, at least or up to about 6, at least or up to about 7, at least or up to about 8, at least or up to about 9, or at least or up to about 10.

In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein. Alternatively or in addition not, the gene regulating moiety can induce epigenetic modification (or epigenome modification) as provided herein.

In some cases, the modification of the polynucleotide sequence or the target gene, as provided herein, can inactivate the polynucleotide sequence or the target gene. For example, modification of the polynucleotide sequence or the target gene can repress or reduce expression and/or activity level of the polynucleotide sequence or the target gene. In some cases, the modification of the polynucleotide sequence or the target gene, as provided herein, can activate the polynucleotide sequence or the target gene. For example, modification of the polynucleotide sequence or the target gene can increase expression and/or activity level of the polynucleotide sequence or the target gene.

In some cases, the modification of the polynucleotide sequence or the target gene, as provided herein, can comprise decreasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 95%, at least or up to about 99%, or about 100% (e.g., as compared to a control that, for example, lacks the modification).

In some cases, the modification of the polynucleotide sequence or the target gene, as provided herein, can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, or at least or up to about 100-fold (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1%, at least or up to about 0.2%, at least or up to about 0.3%, at least or up to about 0.4%, at least or up to about 0.5%, at least or up to about 1%, at least or up to about 2%, at least or up to about 3%, at least or up to about 4%, at least or up to about 5%, at least or up to about 10%, at least or up to about 15%, at least or up to about 20%, at least or up to about 30%, at least or up to about 40%, at least or up to about 50%, at least or up to about 60%, at least or up to about 70%, at least or up to about 80%, at least or up to about 90%, at least or up to about 100%, at least or up to about 150%, at least or up to about 200%, at least or up to about 300%, at least or up to about 400%, or at least or up to about 500% (e.g., as compared to a control that, for example, lacks the modification).

In some cases, the modification of the polynucleotide sequence or the target gene, as provided herein, can comprise increasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 1.5-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 11-fold, at least or up to about 12-fold, at least or up to about 13-fold, at least or up to about 14-fold, at least or up to about 15-fold, at least or up to about 20-fold, at least or up to about 30-fold, at least or up to about 40-fold, at least or up to about 50-fold, at least or up to about 100-fold, at least or up to about 200-fold, at least or up to about 300-fold, at least or up to about 400-fold, at least or up to about 500-fold, or at least or up to about 1,000-fold (e.g., as compared to a control that, for example, lacks the modification).

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least or up to about 1 gate moiety, at least or up to about 2 gate moieties, at least or up to about 3 gate moieties, at least or up to about 4 gate moieties, at least or up to about 5 gate moieties, etc.) and/or a gene regulating moiety (e.g., at least or up to about 1 gene regulating moiety, at least or up to about 2 gene regulating moieties, at least or up to about 3 gene regulating moieties, at least or up to about 4 gene regulating moieties, at least or up to about 5 gene regulating moieties, at least or up to about 6 gene regulating moieties, at least or up to about 7 gene regulating moieties, at least or up to about 8 gene regulating moieties, at least or up to about 9 gene regulating moieties, at least or up to about 10 gene regulating moieties, etc.). A gate moiety as disclosed herein can comprise a guide nucleic acid molecule (gNA) (e.g., at least or up to about 1 gNA molecule, at least or up to about 2 gNA molecules, at least or up to about 3 gNA molecules, at least or up to about 4 gNA molecules, at least or up to about 5 gNA molecules, etc.). A gene regulating moiety as disclosed herein can comprise a gNA (e.g., at least or up to about 1 gNA molecule, at least or up to about 2 gNA molecules, at least or up to about 3 gNA molecules, at least or up to about 4 gNA molecules, at least or up to about 5 gNA molecules, etc.). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some cases, a single gate unit can comprise a single gene regulating moiety per a target gene (e.g., a target endogenous gene). For example, activation of the single gate unit can effect the gene regulating moiety to express a functional guide nucleic acid molecule for binding to and modulating expression/epigenetic profile of the target gene. Alternatively, a single gate unit can comprise a plurality of gene regulating moieties per a target gene for multiplex targeting. For example, the plurality of gene regulating moieties can encode a plurality of different guide nucleic acid molecules with a plurality of different spacer sequences against a common target gene, such that activation of the single gate unit can effect the plurality of gene regulating moieties to express the plurality of different guide nucleic acid molecules (e.g., substantially simultaneously based on the activation of the single gate unit alone) for multiplex targeting and modulation of expression/epigenetic profile of the common target gene.

In some cases, when a heterologous genetic circuit is designed to sequentially modulate expression/epigenetic profile of a plurality of target genes, each of the plurality of target genes may be modulated via a single gene regulating moiety at a given step of the heterologous genetic circuit. In some cases, each of the plurality of target genes may be modulated via multiplex targeting at a given step of the heterologous genetic circuit. In some cases, one target gene may be modulated via a single gene regulating moiety at a given step of the heterologous genetic circuit, while the a different target gene may be modulated via multiplex targeting at a given step of the heterologous genetic circuit.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety (e.g., electromagnetic energy) may activate the initial gate unit without directly binding the at least the portion of the initial gate unit. In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, a vector (or expression cassette) encoding the activatable gNA can comprise an inactivation polynucleotide sequence to render the gNA inactive until activated (e.g., until the inactivation polynucleotide sequence is modified or removed from the vector. For example, the inactivation polynucleotide sequence can encode a self-cleaving polynucleotide molecule (e.g., a ribozyme). Alternatively or in addition to, the inactivation polynucleotide sequence can encode non-canonical transcription termination sequence, as described below. The inactivation polynucleotide sequence can be a part of or adjacent to a region of the vector that encodes (i) a spacer sequence of the gNA, (ii) a scaffold sequence of the gNA, and/or (ii) any linker sequence between the spacer sequence and the scaffold sequence. The vector can comprise at least or up to about 1 inactivation polynucleotide sequence, at least or up to about 2 inactivation polynucleotide sequences, at least or up to about 3 inactivation polynucleotide sequences, at least or up to about 4 inactivation polynucleotide sequences, at least or up to about 5 inactivation polynucleotide sequences, at least or up to about 6 inactivation polynucleotide sequences, at least or up to about 7 inactivation polynucleotide sequences, at least or up to about 8 inactivation polynucleotide sequences, at least or up to about 9 inactivation polynucleotide sequences, or at least or up to about 10 inactivation polynucleotide sequences.

In some cases, the term “proGuide” as generally used herein may refer to such vector (e.g., a plasmid) that encodes the activatable gNA. The proGuide can be an example of a gate moiety. The proGuide can be an example of a gene regulating moiety.

A proGuide can comprise a linker sequence between (i) a domain encoding a spacer sequence of a guide nucleic acid and (ii) a domain comprising a scaffold sequence of the guide nucleic acid, which domain comprising one or more inactivation polynucleotide sequences (e.g., one or more polyT sequences). Alternatively, the proGuide may not comprise a linker sequence between the two domains (i) and (ii).

A proGuide can comprise a target polynucleotide domain at or adjacent to an inactivation polynucleotide sequence (e.g., at or adjacent to 5′ and/or 3′ ends of the inactivation polynucleotide sequences), which target polynucleotide domain can be targeted (e.g., via sequential activation mechanism of the heterologous genetic circuit as provided herein) to modify (e.g., edit, cleave) the inactivation polynucleotide sequence, thereby rendering the proGuide to express an activated guide nucleic acid molecule. The target polynucleotide domain of a proGuide may not exhibit sequence identity to any comparable endogenous polynucleotide sequence in a cell, thereby to avoid inadvertent targeting and modulation of an endogenous target gene.

In some embodiments, the inactivation polynucleotide sequence of the proGuide can be disposed between two target polynucleotide domains, which may or may not be targetable by a common guide nucleic acid sequence. In some cases, the two target polynucleotide domains can be reverse and complementary to one another, such that the inactivation polynucleotide sequence can be modified or cleaved by the same mechanism (e.g., same spacer sequence of a guide nucleic acid molecule).

In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the activatable gNA molecule comprises a non-canonical transcription termination sequence (e.g., a polyX sequence, such as a polyU sequence or a polyT sequence), such that a functional gNA molecule is not expressed until a gene encoding the activatable gNA having the non-canonical transcription termination sequence can be modified (e.g., to remove some or all of the transcription termination sequence). Thus, in absence of the modification of the transcription termination sequence, a non-functional variant (e.g., a non-functional fragment) of the gNA may be expressed. In some embodiments, the gNA can be synthetic. In some embodiments, the gNA can have a fluorescent label attached.

In some cases, a size of the polyT sequence is greater than or equal to a threshold length, wherein the threshold length is sufficient to reduce expression of the guide nucleic acid molecule from the polynucleotide sequence. Accordingly, a plasmid (e.g., a gate moiety or a gene regulating moiety) can encode an inactivated gNA comprising the polyT sequence that is greater than or equal to the threshold length, and editing of such plasmid to reduce the length of the polyT to below the threshold length can permit expression of the gNA in its entirety without early termination, thereby activating the gNA. In some cases, the polyT sequence comprises at least 5 T. In some cases, the polyT sequence comprises at least 7 T. In some cases, the polyT sequence comprises at least 8 T. In some cases, the polyT sequence comprises at least 10 T. In some cases, the polyT sequence comprises between 5 T and 15 T. In some cases, the polyT sequence comprises one or more additional nucleotides that are not T.

In some cases, a gene regulating moiety (e.g., a guide nucleic acid and/or an endonuclease) can be configured to bind to a target polynucleotide sequence operatively coupled to a target gene in a cell. The target gene can comprise an encoding polynucleotide sequence that encodes a target nucleic acid molecule or a target protein. The target polynucleotide sequence can be a part of the encoding polynucleotide sequence. Alternatively, the target polynucleotide sequence may not be a part of the encoding polynucleotide sequence. For example, the target polynucleotide sequence can be upstream of the encoding polynucleotide sequence (e.g., part of a promoter of the encoding polynucleotide sequence, such as a transcription start site (TSS).

As provided herein, when the heterologous genetic circuit is activated to induce a plurality of distinct modulations of a target gene, as provided herein, the plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. Alternatively or in addition to, the distinct modulation of the target gene can be substantially the same (e.g., the same).

The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

As provided herein, the conversion of the plurality of cells of the first cell type into the plurality of cells of the second cell type via use of the heterologous gene modulator (e.g., as part of a heterologous genetic circuit) can be performed in absence of one or more heterologous factors that are normally required to induce a comparable conversion in absence of the heterologous gene modulator. In the comparable conversion, the one or more heterologous factors can be exogenously added to a cell culture medium, or can be expressed in the cells. In some cases, for converting differentiated cells into stem cells (e.g., iPSCs), the one or more heterologous factors can include one or more reprogramming factors such as Oct4, Sox2, Nanog, Lin28, L-Myc, Klf4, and/or SV40LT.

In some cases, use of the heterologous genetic circuit as disclosed herein can be used to convert a plurality of cells of a first cell type into a plurality of a second cell type. In some cases, the first plurality of cells can be differentiated cells, and the second plurality of cells can be stem cells (e.g., iPSCs). Alternatively, the first plurality of cells can be stem cells, and the second plurality of cells can be differentiated cells. In some cases, a differentiated cell is converted into a stem cell (e.g., iPSC). In some cases, a stem cell is converted into a different type of stem cell. In some cases, a stem cell is converted into a differentiated cell. In some cases, a differentiated cell is converted into a different type of differentiated cell. In some cases a differentiated cell is a terminally differentiated cell.

A terminally differentiated cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, an secretory cell, a blood cell, a germ cell, a nurse cell, a storage cell, an enteroendocrine cell, a pituitary cell, a neurosecretory cell, a duct cell, an odontoblast, a cementoblast, a glial cell, or an interstitial cell.

Non-limiting examples of such cell can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffin cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme—rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells (e.g., CD34+ cells, peripheral blood mononuclear cells), Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

A stem cell can comprise an induced pluripotent stem cell (iPSC), an embryonic stem cell (ESC), a mesenchymal stem cell (MSC), a hematopoietic stem cell (HSC) (e.g., from cord blood), a muscle stem cell, a neural stem cell, an epithelial stem cell, an epidermal stem cell, a mammary stem cell, an intestinal stem cell, a neural crest stem cell, or a testicular stem cell.

A stem cell such as induced pluripotent stem cell can be identified by cell surface markers. Non-limiting examples of iPSC cell surface markers include 5T4, ABCG2, Activin RIB/ALK4, Activin RIIB, Alkaline Phosphatase/ALPL, B18R, E-Cadherin, Cbx2, CD9, CD30/TNFRSF8, CD117, CDX2, CDH3, CHD1, Cripto, DNMT3B, DPPA2, DPPA4, DPPA5, ESG1, EpCAM, TROP1, ERR beta, NR3B2, ERVMER34-1, ESGP, FBXO15, FGF4, FGF5, FOXD3, GCTM2, GBX2, GCNF, GDF3, CD49, CD29, KLF4, KLF5, L1TD1, Lefty1, LIN28, LIN41, c-Maf, c-Myc, Nanog, OCT3, OCT4, Polocalyxin, SMAD2, SMAD3, SOX2, SSEA1, SSEA3, SSEA4, STAT3, SUZ12, TBX2, TBX3, TBX5, TERT, TEX19, THAP11, TRA-1-60, TRA-1-81 TROP, UTF1, VISTA, and/or ZIC3.

Various aspects of the present disclosure provide for contacting the first plurality of cells with a heterologous gene modulator exhibiting specific binding to a target gene that is derived from a non-mammalian genome (e.g., a non-human genome), to effect conversion of the first plurality of cells of a first cell type into a plurality of cells of a second cell type (e.g., from a differentiated cell to a stem cell). In some embodiments, the target gene can be (i) derived from a virus (e.g., a DNA virus, a RNA virus, or a reverse transcribing virus (e.g., a retrovirus or a pararetrovirus)) and (ii) integrated in a non-viral genome (e.g., a mammalian genome, such as a human genome). In some cases, the target gene can be derived from a retrovirus, such as, for example, a human endogenous retrovirus (HERV) gene. The target gene can comprise at least a portion of the HERV gene. The at least the portion of the HERV gene can comprise one or more members selected from the group consisting of one or more long terminal repeats (LTRs) (e.g., U3, R, U5), a Group-specific antigen (GAG) gene (e.g., matrix (MA) domain, capsid (CA) domain, nucleocapsid (NC) domain), a protease (PR) gene, a polymerase (POL) gene (e.g., reverse transcriptase (RT), Ribonuclease H (RH), Integrase (IN)), and an envelope (ENV) gene (e.g., surface component (SU), trans-membrane component (TM)). The LTR as disclosed herein can be an LTR that is disposed towards the upstream of the target gene, e.g., 5′ LTR. Non-limiting examples of the LTR of a HERV can include LTR5HS, LTR5A, and LTR5B. Non-limiting examples of LTR5HS can include LTR5HS-1, LTR5HS-2, LTR5HS-3, LTR5HS-4, LTR5HS-5, LTR5HS-6, LTR5HS-7, LTR5HS-8, LTR5HS-9, LTR5HS-10, LTR5HS-11, and LTR5HS-12.

LTR5HS is the regulatory element subgroup which regulates all human HERV-K elements. A LTR5HS element can be regulated through DNA hypermethylation. Alternatively, a LTR5HS element can be regulated through DNA hypomethylation or DNA demethylation.

In some cases, the heterologous gene modulator exhibits specific binding to a gene (or a target gene) encoding a mobile genetic element (MGE). MGEs are segments of genetic material that can move around withing a genome or which can be transferred from one species or replicon to another. Non-limiting examples of MGEs are plasmids, transposons, integrons, viral agents, and introns.

Transposons, also known as jumping genes, are a group of mobile genetic elements that are DNA sequences. Transposons can move to different places within a genome. Alternatively, some transposons are always kept at particular insertion sites within a genome. Transposons are divided into two main groups: retrotransposons and DNA transposons. Retrotransposons are often found in eukaryotes. Non-limiting examples of retrotransposons include long terminal repeats, or LTRs, and non-long terminal repeats, or non-LTRs (e.g., long interspersed nuclear elements (LINEs) and short interspersed nuclear elements (SINEs)). DNA transposons can be found in both eukaryotes and prokaryotes. Non-limiting examples of DNA transposons include cut and paste DNA transposons, rolling circle DNA transposons (also called helitrons), and self-synthesizing DNA transposons (also called polintons).

Long interspersed nuclear elements (LINEs) are transposable elements that are typically about 7000 base pairs in length. LINEs often function by being transcribed into mRNA and translated into proteins that act as a reverse transcriptase to integrate DNA copies into the genome at a new site. Non-limiting examples of LINEs include LINE-1, L1H, RTE, Tad, and CRE.

Short interspersed nuclear elements (SINEs) are transposable elements that are typically about 100-700 base pairs in length. SINEs are often lineage specific. One example of a SINE is an Alu element, which are primate-specific repeats. Alu elements about 11% of the human genome. Alu elements are typically around 300 base pairs in length. Alu elements have been implicated in several human diseases such as cancer.

In some cases, the heterologous gene modulator exhibits specific binding to a gene (or a target gene) encoding (or comprising or operatively coupled to, as used interchangeably herein) embryo genome activation (EGA)-enriched Alu-motif (EEA). EEAs, or EEA motifs, are Alu elements enriched near genes involved in embryo genome activation. EEA motif-associated mechanisms are implicated in cellular reprograming through the activation of genes such as NANOG and REX1.

Long terminal repeats are pairs of DAN sequences, hundreds of bases in length, which occur in eukaryotic genomes at either end of retrotransposons or endogenous retroviruses. LTRs typically encode a reverse transcriptase and an integrase, allowing the retrotransposon to be copied and inserted at a different location. Non-limiting examples of LTRs include HERV, MLT2A1, and MLT2A2.

In some cases, the heterologous gene modulator exhibits specific binding to a gene (or a target gene) encoding human endogenous retroviruses, or HERVs. HERVs are stable elements in DNA that are left-over from ancient infections that affected primate ger lines in the past 100 million years. HERVs and their genetic products, which include RNA, cytosolic DNA, and proteins, are able to modulate and be influenced by the immune system. HERVs have been implicated with assisting the immune system in defending against exogenous infections. Non-limiting examples of HERVs include HERV-H (e.g., RTVL-L, RGH), HERV-F, HERV-R (e.g., ERV9, ERV3), HERV-P (e.g., HuERS-P, HuRRS-p), HERV-L, HERV-I (e.g., RTVL-I), HERV-IP-T47D (e.g, ERV-FTD), HERV-K (e.g., HML-1, HML-1.1, HML-2, HERV-K10, HERV-K-HTDV, HML-3, HML-3.1, HML-4, HERV-K-T47D, HML-5, HERV-K-NMWV2, HML-6, HML-6p, HML-7, KERV-K-NMWV7, HML-8, HERV-K-NMWV3, HML-9, HERV-K-NMWV9, HML-10), HERV-W, HERV-E (e.g., 4-1, ERVA, NP-2), 51-1, RRHERV-I, HERV-T (e.g., S71, CRTK1, CRTK6), and ERV-FRD.

In some cases, the heterologous gene modulator exhibits specific binding to a gene (or a target gene) encoding HERV-K. HERV-K is a family of 30-50 sequences that are highly conserved in primates. HERV-K proteins have protease, but not reverse transcriptase enzymatic activity. HERV-K is often expressed at low steady-state levels in many human tissues and tumors.

In some cases, the heterologous gene modulator comprises an endonuclease (e.g., Cas9) that is capable of forming a complex with the nucleic acid molecule. In some cases, the heterologous gene modulator further comprises a gene regulator. A gene regulator can be a gene activator, a DNA binding protein that has positive control over gene expression. Non-limiting examples of gene activators can include VP16, VP64, p65, p53, E2F1, TAT, E2A, NFAT, GAL4, CGN4, HAP1, MLL, TRG3, GLN3, OAF1, PIP2, PDR1, PDR3, PHO4, LEU3, RTA, and VP64-p65-RTA fusion (VPR). A gene activator can be coupled to the endonuclease. gene regulator can be a gene repressor, a DNA binding protein that has negative control over gene expression. Non-limiting examples of gene repressors can be a tetracycline repressor, an AMP early repressor (ICER), a Kruppel-associated box (KRAB), a YY1 glycine rich repressor, a Sp 1-like repressor, an E(spI) repressor, an IκB repressor, or MeCP2.

Various aspects of the present disclosure provide engineered cells that are programmed to induce a desired expression and/or activity level (or profile thereof) of one or more target genes in a cell.

One or more target genes targeted by a heterologous gene modulator as disclosed herein can comprise a cell de-differentiation factor (e.g., a Yamanaka factor). In some cases, the one or more target genes can comprise Oct3, Oct4, Sox2, Klf4, c-Myc, miR-302, miR-307, HERV-K, EEA, ZSCAN4, DUX4, OTX2, ABCE1, COL5A1, GAL4NT13, DUXA, DUXB, ARGFX, CPHX1, CPHX2, TPRX1, DPPA3, NASP, ATP2B1, NFAT, H1FOO, and/or CAMKII.

In some cases, the one or more target genes of the heterologous genetic circuit can comprise OCT4, SOX2, KLF4, and/or MYC. In some cases, the heterologous genetic circuit can be preconfigured such that (i) regulation of the expression level or epigenetic profile level of a first target endogenous gene from OCT4, SOX2, KLF4, and/or MYC occurs prior to (ii) regulation of the expression level or epigenetic profile level of a second target endogenous gene from OCT4, SOX2, KLF4, and/or MYC. For example, the heterologous genetic circuit can be programmed such that activation of a first gate unit preconfigured for targeting and modulating expression/epigenetic level of the first target endogenous gene can occur prior to activation of a second gate unit preconfigured for targeting and modulating expression/epigenetic level of the second target endogenous gene (e.g., in absence of human intervention or any secondary activation of the heterologous genetic circuit after activation of the first gate unit or after activation of the heterologous genetic circuit). The first target endogenous gene can be OCT4, and the second target endogenous gene can be SOX2, KLF4, and/or MYC. The first target endogenous gene can be SOX2, and the second target endogenous gene can be OCT4, KLF4, and/or MYC. The first target endogenous gene can be KLF4, and the second target endogenous gene can be OCT4, SOX2, and/or MYC. The first target endogenous gene can be MYC, and the second target endogenous gene can be OCT4, SOX2, and/or KLF4. Without wishing to be bound by theory, directing and controlling sequential modulation of target genes while minimizing intervention (e.g., human intervention) or disruption of the cells can (i) maintain cellular characteristics of the cells, such as viability, proliferative capacity, and/or therapeutic efficacy and/or (ii) enhance desired outcome (e.g., reprogramming of cells as provided herein) of cellular engineering.

The one or more target genes can comprise HERV (e.g., HERV-K), POU family transcription factor (e.g., Oct4), KLF4, MYC, SOX2, EEA, and miR-302. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-63, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-63, or a complementary sequence thereof (e.g., with uracil-to-thymine conversion).

The one or more target genes can comprise HERV (e.g., HERV-K). In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-12, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 1-12, or a complementary sequence thereof (e.g., with uracil-to-thymine conversion).

The one or more target genes can comprise POU family transcription factor (e.g., Oct4). OCT4 is a transcription factor in the POU family. OCT4 is involved in the self-renewal of undifferentiated embryonic stem cells and is a commonly used marker for undifferentiated cells. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 13-17, or a complementary sequence thereof. In some cases, a spacer sequence of a guide nucleic acid (e.g., a guide RNA) as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 13-17, or a complementary sequence thereof.

The one or more target genes can comprise SOX2. SOX2 is involved in the self-renewal of undifferentiated embryonic stem cells and is a commonly used marker for undifferentiated cells. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 42-58, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 42-58, or a complementary sequence thereof (e.g., with uracil-to-thymine conversion).

The one or more target genes can comprise MYC (or C-Myc). C-Myc is a regulator gene and a transcription factor that is involved in cell proliferation. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 30-41, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 30-41, or a complementary sequence thereof (e.g., with uracil-to-thymine conversion).

The one or more target genes can comprise KLF4. KLF4, also known as Kruppel-like factor 4, is a zinc finger transcription factor that is involved in the regulation of proliferation, differentiation, apoptosis, and somatic cell reprogramming. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 25-29, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 25-29, or a complementary sequence thereof (e.g., with uracil-to-thymine conversion).

The one or more target genes can comprise miR-302. miR-302 is a polycistronic miRNA cluster that can induce and maintain pluripotency. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 18-24, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 18-24, or a complementary sequence thereof (e.g., with uracil-to-thymine conversion).

The one or more target genes can comprise EEA. In some cases, a spacer sequence of a guide nucleic acid (e.g., a guide RNA) against a target gene as provided herein can comprise a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 59-63, or a complementary sequence thereof. In some cases, a heterologous gene modulator as provided herein can exhibit specific binding to a target gene that comprises a polynucleotide sequence (e.g., a consecutive polynucleotide sequence) that exhibits at least or up to about 50%, at least or up to about 55%, at least or up to about 60%, at least or up to about 65%, at least or up to about 70%, at least or up to about 75%, at least or up to about 80%, at least or up to about 85%, at least or up to about 86%, at least or up to about 87%, at least or up to about 88%, at least or up to about 89%, at least or up to about 90%, at least or up to about 91%, at least or up to about 92%, at least or up to about 93%, at least or up to about 94%, at least or up to about 95%, at least or up to about 96%, at least or up to about 97%, at least or up to about 98%, at least or up to about 99%, or substantially about 100% sequence identity to one or more members selected from SEQ ID NOs: 59-63, or a complementary sequence thereof (e.g., with uracil-to-thymine conversion).

In some cases, contacting a plurality of cells with the heterologous gene modulator can de-differentiate cells into iPSCs. Using a heterologous gene modulator as disclosed herein can generate at least about 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, 1×10⁸, 2×10⁸, 5×10⁸, 1×10⁹, 2×10⁹, 5×10⁹, 1×10¹⁰, 2×10¹⁰, 5×10¹⁰, 1×10¹⁵, 2×10¹⁵, 5×10¹⁵, or more iPSCs from at most about 1×10⁶, 9×10⁵, 8×10⁵, 7×10⁵, 6×10⁵, 5×10⁵, 4×10⁵, 3×10⁵, 2×10⁵, 1×10⁵, 5×10⁴, 2×10⁴, 1×10⁴, or less terminally differentiated cells.

Such generation of iPSCs by using the heterologous gene modulator as disclosed herein can be achieved within the span of at most about 1 day, at most about 2 days, at most about 3 days, at most about 4 days, at most about 5 days, at most about 6 days, at most about 7 days, at most about 8 days, at most about 9 days, at most about 10 days, at most about 11 days, at most about 12 days, at most about 13 days, at most about 14 days, at most about 15 days, at most about 20 days, at most about 25 days, at most about 30 days, at most about 35 days, at most about 40 days, at most about 45 days, at most about 50 days, at most about 55 days, at most about 60 days, or at most about 65 days.

The conversion of the plurality of cells of the first cell type into the plurality of cells of the second cell type (e.g., reprogramming of differentiated cells into stem cells, such as pluripotent stem cells) can be characterized by having a conversion efficiency (or a rate of conversion) of at least or up to about 5%, at least or up to about 10%, at least or up to about 15%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 99%, or about 100%.

In some cases, the heterologous gene modulator and the additional heterologous gene modulator are introduced to a cell at substantially the same time. In some cases, the heterologous gene modulator is introduced prior to the additional heterologous gene modulator. In some cases, the heterologous gene modulator is introduced after the additional heterologous gene modulator.

In some cases, the heterologous gene modulator and the additional heterologous gene modulator may or may not be part of the same heterologous genetic circuit. The heterologous gene modulator and the additional heterologous gene modulator may be part of different heterologous genetic circuits. Alternatively, the heterologous gene modulator and the additional heterologous gene modulator both may not be a part of any heterologous genetic circuit.

In some cases, a cell or plurality of cells can additionally be contacted by an inhibitor. In some cases, a cell or plurality of cells can be contacted by an inhibitor prior to introduction of the heterologous gene modulator. Alternatively, a cell or plurality of cells can be contacted by an inhibitor substantially at the same time as introduction of the heterologous gene modulator. Alternatively, a cell or plurality of cells can be contacted by an inhibitor subsequent to introduction of the heterologous gene modulator. An inhibitor is a gene whose presence prevents the expression of another gene at a different locus. An inhibitor can be a p53 inhibitor, e.g., in the form of a small molecule drug or a p53 short hairpin RNA (shRNA).

In some cases, a target gene may be subjected to one (e.g., a single) gene modulation (e.g., a single targeted activation step). In some cases, a target gene may be subjected to at least two heterologous gene modulators comprising a first modulator making a first modulation and a second (or additional) modulator making a second (or additional) modulation. Timing of the first modulation and the second modulation can be controlled (e.g., as predetermined by the design of the heterologous genetic circuit). For example, the onset of the second modulation (e.g., by at least a portion of the second gate unit, such as the second gene regulation moiety) can occur subsequent to the onset of the first modulation (e.g., by at least a portion of the first gate unit, such as the first gene regulating moiety) by at least about 1 second, at least about 2 seconds, at least about 3 seconds, at least about 4 seconds, at least about 5 seconds, at least about 6 seconds, at least about 7 seconds, at least about 8 seconds, at least about 9 seconds, at least about 10 seconds, at least about 20 seconds, at least about 30 seconds, at least about 40 seconds, at least about 50 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 20 hours, at least about 1 day, at least about 2 days, at least about 3 days, at least about 4 days, at least about 5 days, at least about 6 days, at least about 7 days, at least about 8 days, at least about 9 days, or at least about 10 days. The onset of the second modulation (e.g., by at least a portion of the second gate unit, such as the second gene regulation moiety) can occur subsequent to the onset of the first modulation (e.g., by at least a portion of the first gate unit, such as the first gene regulation moiety) by at most about 10 days, at most about 9 days, at most about 8 days, at most about 7 days, at most about 6 days, at most about 5 days, at most about 4 days, at most about 3 days, at most about 2 days, at most about 1 day, at most about 20 hours, at most about 10 hours, at most about 9 hours, at most about 8 hours, at most about 7 hours, at most about 6 hours, at most about 5 hours, at most about 4 hours, at most about 3 hours, at most about 2 hours, at most about 1 hours, at most about 50 minutes, at most about 40 minutes, at most about 30 minutes, at most about 20 minutes, at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 1 minutes, at most about 50 seconds, at most about 40 seconds, at most about 30 seconds, at most about 20 seconds, at most about 10 seconds, at most about 9 seconds, at most about 8 seconds, at most about 7 seconds, at most about 6 seconds, at most about 5 seconds, at most about 4 seconds, at most about 3 seconds, at most about 2 seconds, or at most about 1 second.

In some cases, a number of gate units that need to be activated (e.g., sequentially activated) between the activation of the first modulation by the first gate unit and the later activation of the second modulation by the second gate unit can at least in part determine (e.g., substantially determine) the timing between the first modulation and the second modulation. Upon activation of the first modulation of the target gene by the first gate unit, at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more additional gate units may need to be activated (e.g., sequentially activated) to activate the second gate unit for inducing the second modulation. Upon activation of the first modulation of the target gene by the first gate unit, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 additional gate units may need to be activated (e.g., sequentially activated) to activate the second gate unit for inducing the second modulation.

The outcome of a cell can comprise the regulation of a plurality of target genes. For example, the outcome can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The outcome can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic modulator and/or (ii) the activating moiety of the heterologous genetic modulator.

The first distinct modulator can induce a change (e.g., increase or decrease) in the expression and/or activity level of the target gene (e.g., HERV-K) by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. The first distinct modulation can induce a change (e.g., increase or decrease in the expression and/or activity level of the target gene (e.g., HERV-K) by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

The first distinct modulation as disclosed herein (e.g., induced by the first gate unit) can induce a change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The first distinct modulation can induce a change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Subsequently, a second distinct modulation as disclosed herein (e.g., induced by the second gate unit) can induce an additional change (e.g., increase, decrease, or selective attenuation) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce an additional change (e.g., increase or decrease) in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%.

The additional change via the second distinct modulation can induce an additional change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The second distinct modulation can induce an additional change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

The additional change via the second distinct modulation can occur when the expression and/or activity level of the target gene reaches a target level via action of the first distinct modulation, e.g., by design of the heterologous genetic circuit.

The additional change via the second distinct modulation can occur when the expression and/or activity level of the target gene is changed (e.g., increased or decreased) via action of the first distinct modulation by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The additional change via the second distinct modulation can occur when the expression and/or activity level of the target gene is changed (e.g., increased or decreased) via action of the first distinct modulation by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Alternatively, or in addition to, a second distinct modulation as disclosed herein (e.g., induced by the second gate unit) can induce a change (e.g., increase or decrease) in the expression and/or activity level of an additional target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce a change (e.g., increase or decrease) in the expression and/or activity level of the additional target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. A cell can comprise a prokaryotic cell, a eukaryotic cell, or an artificial cell.

The stem cells (e.g., iPSCs) generated by the systems and methods of the present disclosure can be capable of being differentiated into a cell type found in one or more tissues. Non-limiting examples of such tissues can include skin, heart, lung, kidney, bone, cartilage, bone marrow, breast, pancreas, liver, muscle, smooth muscle, bladder, gall bladder, colon, intestine, brain, prostate, esophagus, thyroid, blood, serum, reproductive organ, etc.

The engineered cell (e.g., the engineered iPSC) or any further engineered variant thereof (e.g., a differentiated byproduct of such iPSCs) of the present disclosure can be used (e.g., administered) to treat a subject in need thereof. The subject can have or can be suspected of having a condition, such as a disease (e.g., cancer). A cell (e.g., a differentiated cell) can be obtained from the subject and such cell can be cultured ex vivo and genetically modified to generate an iPSC as disclosed herein. Subsequently, the engineered iPSC can be administered to the subject for adaptive immunotherapy. Thus, the engineered cell or any further engineered variant thereof can be autologous to the subject in need thereof. Alternatively, the engineered cell or any further engineered variant thereof can be allogeneic to the subject (e.g., allogeneic stem cell transplantation, allogeneic adoptive immunotherapy, etc.).

The engineered cells or any further engineered variant thereof as disclosed herein can be administered to the subject prior to, concurrently with, or subsequent to activation of the heterologous genetic circuit(s) and/or heterologous genetic modulators in the engineered stem cells. For example, the engineered cells or any further engineered variant thereof can be activated subsequent to being administered into the subject, e.g., by administering to the subject an activator of the heterologous genetic circuit(s).

The subject can be treated (e.g., administered with) a population of engineered cells (e.g., engineered iPSCs) or any further engineered variant thereof of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses. Alternatively, or in addition to, the subject can be treated (e.g., administered with) a population of engineered cells (e.g., engineered iPSCs) or any further engineered variant thereof of the present disclosure for at least about 1 week, at least about 2 weeks, at least about 3 weeks, at least about 4 weeks, at least about 5 weeks, at least about 6, weeks, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, at least about 30 years, at least about 40 years, at least about 50 years, at least about 60 years, at least about 70 years, at least about 80 years, at least about 90 years, or at least about 100 years.

Any one of the methods disclosed herein can be utilized to treat a target cell, a target tissue, a target condition, or a target disease of a subject.

Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

The target disease of the subject can be cancer or tumor. Non-limiting examples of cancer can include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

The present disclosure also provides a composition comprising the engineered genetic modulators and/or the engineered genetic circuits as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered genetic modulators and/or the engineered genetic circuits. Alternatively or in addition to, the activator(s) can be in a different and separate composition from the engineered genetic modulators and/or the engineered genetic circuits.

### EXAMPLES

### Example 1: Heterologous Genetic Circuits for Converting Cell Type Via Modulation of Endogenous Genes

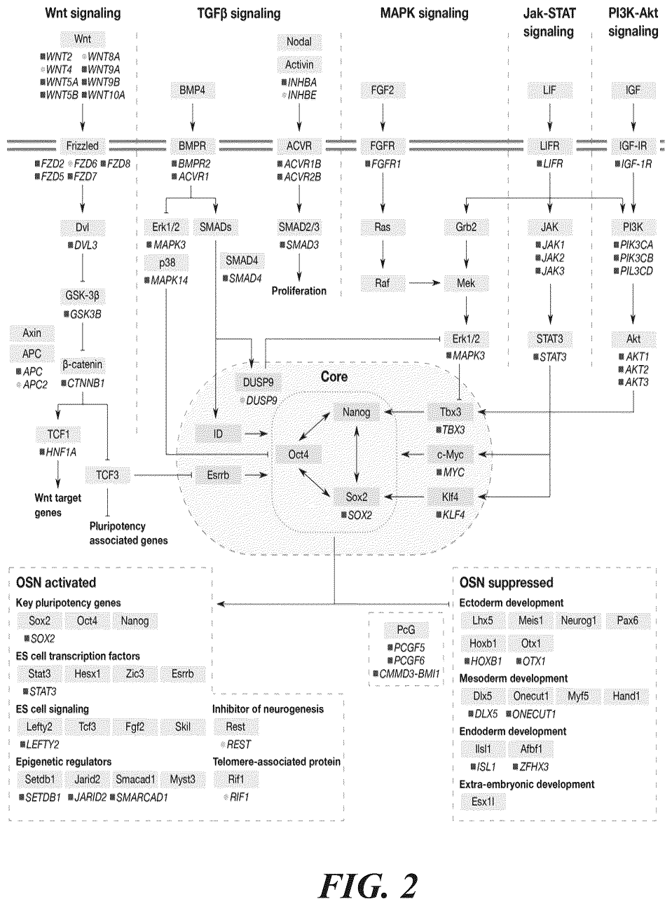

Modulation of (e.g., regulating expression or activity of) one or more endogenous genes can be utilized to convert cells of a first cell type (first plurality of cells) into a plurality of cells of a second cell type (second plurality of cells). In some cases, such modulation can convert differentiated cells (e.g., terminally differentiated cells such as primary fibroblasts) into more stem-like cells (e.g., induced pluripotent stem cells or “iPSCs”). In some cases, the one or more genes can include one or more members from the pluripotency regulating gene network (PGRN) genes, as shown in FIG. 2. Alternatively or in addition to, the one or more genes may not be a PGRN gene (e.g., human endogenous retrovirus (HERV), embryo genome activation (EGA)-enriched Alu-motif (EEA), etc.). In some cases, HERV can comprise HERV-K. In some cases, targeting HERV-K can comprise targeting a regulatory element of HERV-K, such as LTR5HS.

In some cases, the one or more genes can each be modulated once or multiple times (e.g., sequentially). In some cases, the one or more genes can comprise a plurality of different genes, and the plurality of different genes can be modulated substantially at the same time or sequentially.

In some cases, one or more heterologous gene modulators can be used to induce the modulation of the one or more endogenous genes, to effect the conversion. For example, a heterologous gene modulator can include a guide nucleic acid exhibiting specific binding to an endogenous gene (e.g., coding region or non-coding region), which guide nucleic acid can form a complex with a Cas protein or a variant thereof, to induce such modulation. In some cases, the heterologous gene modulator(s) can be part of a heterologous genetic circuit, as provided herein.

### Heterologous Genetic Circuit

Heterologous genetic circuits (HGCs) can be designed, such that each heterologous genetic circuit (e.g., except for a control heterologous genetic circuit) can sequentially activate a plurality of heterologous gene modulators to target a plurality of endogenous genes in primary fibroblasts), to effect conversion (e.g., reprogramming) of the primary fibroblasts into iPSCs. TABLE 1 shows a library of different HGCs for targeting various endogenous genes. The endogenous genes include EEA, Oct4 (O), Klf4 (K), c-Myc (M), Sox2 (S), and miR-302 (e.g., a suppressor of p53; denoted herein as “miRNA”).

A HGC can be designed such that a guide nucleic acid comprising a spacer sequence exhibiting specific binding to a designated target endogenous gene can be activated (e.g., expressed) at the corresponding step. Examples of spacer sequences against the endogenous genes are provided in TABLE 2. The guide nucleic acids are also designed with a scaffold sequence to permit formation of a complex with a Cas-based gene activator (e.g., dCas9-VPR), to specifically bind and activate the target gene(s).

In some cases, one guide nucleic acid sequence with one spacer sequence against a target gene can be used to modulate the target gene. In some cases, a plurality of (or a pool of) nucleic acid sequences with different spacer sequences against a common target gene can be used to modulate the target gene (e.g., multiplexing).

Cells (e.g., primary fibroblasts) can be transfected with the HGC plasmids and a gene encoding the Cas-based gene activator. Subsequently, cells can be transfected with an activator of each HGC, thereby to activate the HGC and trigger the sequential gene modulations.

In some cases, the cells can also be transfected with additional gene regulators that are not part of the HGC. For example, the cells (e.g., primary fibroblasts) can be transfected with a gene encoding a small hairpin RNA (shRNA) against p53 (denoted herein as “p53”), e.g., to facilitate or enhance facilitation of modulation of p53.

TABLE 1
Heterologous Genetic Circuits.

| Condition # | Step 1 | Step 2 | Step 3 | Step 4 |
|---|---|---|---|---|
| 1 | Cells only | | | |
| 2 | Nucleofection only | | | |
| 3 | GFP + core cascade | | | |
| 4 | HERV, EEA, p53 | | | |
| 5 | HERV, O, K, M, S, p53, EEA | | | |
| 6 | HERV, EEA, p53 | O, K, M, S | | |
| 7 | HERV, EEA, p53 | blank | O, K, M, S | |
| 8 | HERV, EEA, p53 | blank | blank | O, K, M, S |
| 9 | HERV, EEA, p53 | O, K, M | S | S |
| 10 | HERV, EEA, p53, O | K, M | S | S |
| 11 | HERV, EEA, p53, O, K | M | MS | S |
| 12 | HERV EEA, p53, O, K, M | S | S | S |
| 13 | HERV, p53 | | | |
| 14 | EEA, HERV, miRNA, p53 | | | |
| 15 | EEA, HERV, miRNA, p53 | O, K, M | S | S |
| 16 | EEA, HERV, miRNA, p53 | blank | OKM | S |
| 17 | EEA, HERV, miRNA, p53, O | K, M | S | S |
| 18 | EEA, HERV, miRNA, p53, O, K | M | MS | S |
| 19 | EEA, p53 | | | |
| 20 | O, K, M, S, p53, EEA | | | |
| 21 | EEA, p53 | O, K, M, S | | |
| 22 | EEA, p53 | blank | O, K, M, S | |
| 23 | EEA, p53 | blank | blank | O, K, M, S |
| 24 | EEA, p53 | O, K, M | S | S |
| 25 | EEA, p53, O | K, M | S | S |
| 26 | EEA, p53, O, K | M | M, S | S |
| 27 | EEA, p53, O, K, M | S | S | S |

TABLE 2
Heterologous Genetic Modulators (e.g., guide nucleic acids)

| Name of | Name of | Spacer Sequence | |
|---|---|---|---|
| target gene | guide RNA | of guide RNA | SEQ ID NO |
| HERV-K | LTR5HS-1 | UACUAAGGGAACUCAG | 1 |
| HERV-K | LTR5HS-2 | AAAACCGCCUUAGGGC | 2 |
| HERV-K | LTR5HS-3 | AAAACUGCCUUAGGGC | 3 |
| HERV-K | LTR5HS-4 | AGCCCGACACCCGUAA | 4 |
| HERV-K | LTR5HS-5 | AGGAGAAAACCACCUU | 5 |
| HERV-K | LTR5HS-6 | UACCAUCUCGGAGAGG | 6 |
| HERV-K | LTR5HS-7 | AACCUUGGACAAUACC | 7 |
| HERV-K | LTR5HS-8 | UAAAGGGUCUGUGCUG | 8 |
| HEERV-K | LTR5HS-9 | GACAUUCCAUUGCCCA | 9 |
| HERV-K | LTR5HS-10 | AUAGAGAAAGAAAAGU | 10 |
| HERV-K | LTR5HS-11 | GAAACGCCCACAGGUG | 11 |
| HERV-K | LTR5HS-12 | AAGCUUUCUUGCUUAG | 12 |
| Oct4 | Oct4-1 | ACUGGCGCCCCUCC | 13 |
| Oct4 | Oct4-2 | AGAAACUGAGGCGA | 14 |
| Oct4 | Oct4-3 | GGCAAUGGUGUCUG | 15 |
| Oct4 | Oct4-4 | GGGGACCUGCACUG | 16 |
| Oct4 | Oct4-5 | GUGCCAGAGGUCUG | 17 |
| miR-302 | miR-302-1 | AAGAAUAGUAUAAAUAGAAG | 18 |
| miR-302 | miR-302-2 | AUCUCAGAGAAUCAUUACAA | 19 |
| miR-302 | miR-302-3 | AGGGAAUGUAUGAUCCUGGG | 20 |
| miR-302 | miR-302-4 | AAAAGGAUCCAGACCCACCC | 21 |
| miR-302 | miR-302-5 | UUUAAGAGGAAGAUAUCUUG | 22 |
| miR-302 | miR-302-6 | AUGCCAUCAAACAAGCAGAU | 23 |
| miR-302 | miR-302-7 | CAAUGCCUUUCUCGGCUCAG | 24 |
| KLF4 | KLF4-1 | UAAGGAACGCGCGC | 25 |
| KLF4 | KLF4-2 | UGUCUGCGGGCGCG | 26 |
| KLF4 | KLF4-3 | CGGGCUUCGAACCC | 27 |
| KLF4 | KLF4-4 | AUAGCAACGAUGGA | 28 |
| KLF4 | KLF4-5 | UCGCUGCGCGACCA | 29 |
| MYC | MYC-1 | GGGGCACACAAAGC | 30 |
| MYC | MYC-2 | CCUGUUCCAGAGCU | 31 |
| MYC | MYC-3 | CAAACCGGCGCCAC | 32 |
| MYC | MYC-4 | GGUACCCGAACCGC | 33 |
| MYC | MYC-5 | CGGGGCAGGAGGGG | 34 |
| MYC | MYC-6 | CAAAGCAGAGGGCG | 35 |
| MYC | MYC-7 | GGUGGGGUACAGAC | 36 |
| MYC | MYC-8 | ACCCAGCCGCUUUA | 37 |
| MYC | MYCN-1 | UGGCGCAGACAAGG | 38 |
| MYC | MYCL | CAGGGCACGGGCGC | 39 |
| MYC | MYCN-2 | CGCAGACAAGGGGG | 40 |
| MYC | MYC-11 | UAAUCUCCGCCCAC | 41 |
| SOX2 | SOX2-1 | GUAAGAGAGGAGAG | 42 |
| SOX2 | SOX2-2 | GCCGCCGGCGGGCC | 43 |
| SOX2 | SOX2-3 | GCGGGGGAGGCCGG | 44 |
| SOX2 | SOX2-4 | CCCAAAGUCCCGGC | 45 |
| SOX2 | SOX2-5 | CACUAAGACUACGU | 46 |
| SOX2 | SOX2-6 | CGCAGGGCUCCGCG | 47 |
| SOX2 | SOX2-7 | GGCAGGCUGGCUCU | 48 |
| SOX2 | SOX2-8 | AAGGUGCGGGGGUG | 49 |
| SOX2 | SOX2-9 | GCGGGCGGGGGGAG | 50 |
| SOX2 | SOX2-10 | CACUUCCUUCGAAA | 51 |
| SOX2 | SOX2-11 | CGCCGCGCGCUGAU | 52 |
| SOX2 | SOX2-12 | UGGCAGGCUGGCUC | 53 |
| SOX2 | SOX2-13 | UCAUCGGCGGCGGC | 54 |
| SOX2 | SOX2-14 | GACCAAUCAGCGCG | 55 |
| SOX2 | SOX2-15 | AGUGUUUGCAAAAG | 56 |
| SOX2 | SOX2-16 | CAUGAAAGGGGGCG | 57 |
| SOX2 | SOX2-17 | CCGGCAGCGAGGCU | 58 |
| EEA | EEA-1 | AAUCCCAGCACUUU | 59 |
| EEA | EEA-2 | GCUACUUGGGAGGC | 60 |
| EEA | EEA-3 | GCCUCCCAAGUAGC | 61 |
| EEA | EEA-4 | GCCUCCCAAAGUGC | 62 |
| EEA | EEA-5 | CCCAGCACUUUGGG | 63 |

### Example 2: Reprogramming of Primary Fibroblasts to iPSCs by Targeting Endogenous Genes (e.g., EEA and/or miR-302)

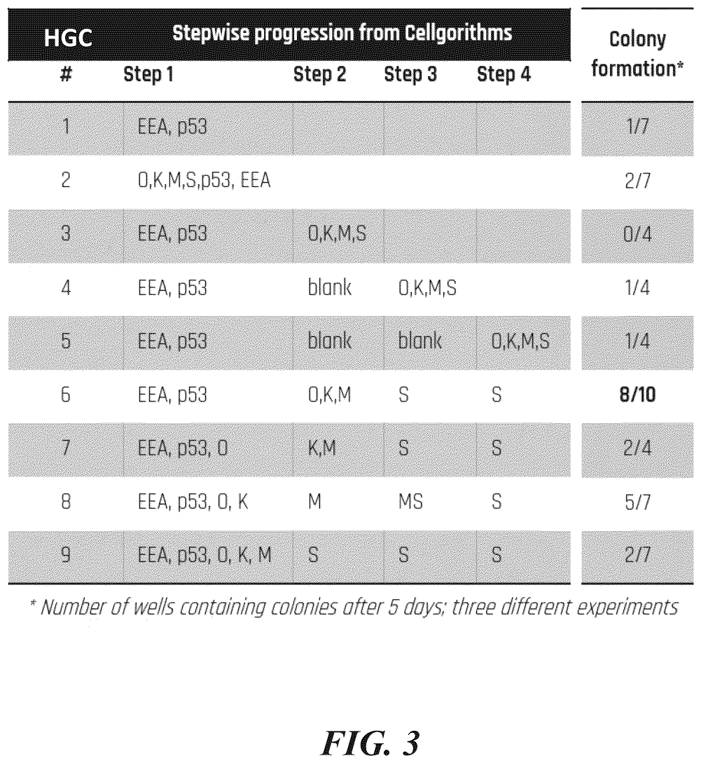

In accordance with Example 1, HGCs were designed to reprogram primary fibroblasts to iPSCs. FIG. 3 shows a library of different HGCs tested, each HGC designed to be capable of targeting one or more endogenous genes sequentially over four steps (step 1, step 2, step 3, and step 4). HGC #1 and 2 were designed as controls, and HGC #3-9 were designed to test sequential targeting of the endogenous genes in various combinations and orders.

Reprogramming of Primary Fibroblasts to iPSCs by Targeting, e.g., EEA and/or miR-302.

Primary fibroblasts were transfected with the respective HGC plasmids or treated as controls (e.g., cells only without any treatment, nucleofection agent alone, etc.) and were subsequently observed (e.g., for 5 days). Each condition had sufficient replicates (e.g., n=4) at one or more time points for assessment (e.g., assessing expression level of the target endogenous genes and/or embryogenic markers such as Nanog via RNA isolation or staining). The cells were also visually assessed for colony formation/expansion, which is indicative of reprogramming of fibroblasts into iPSCs.

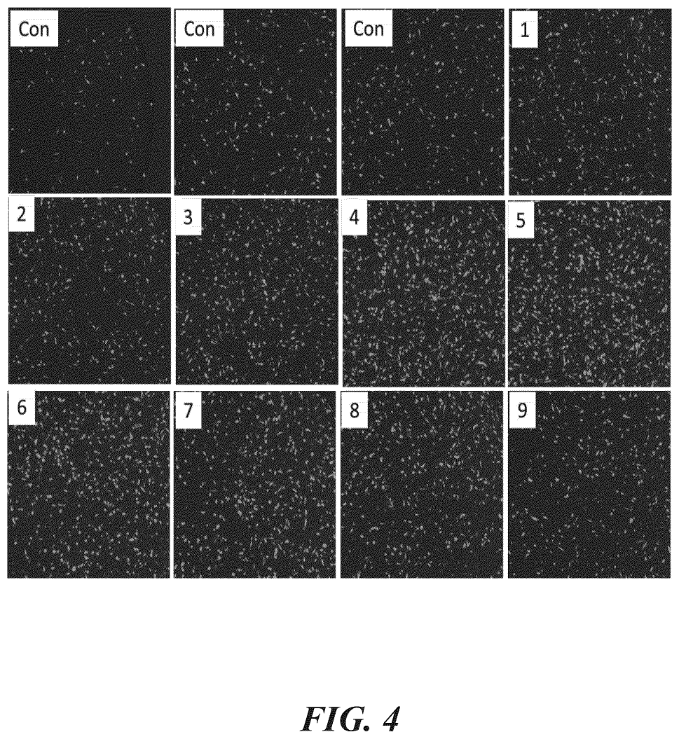

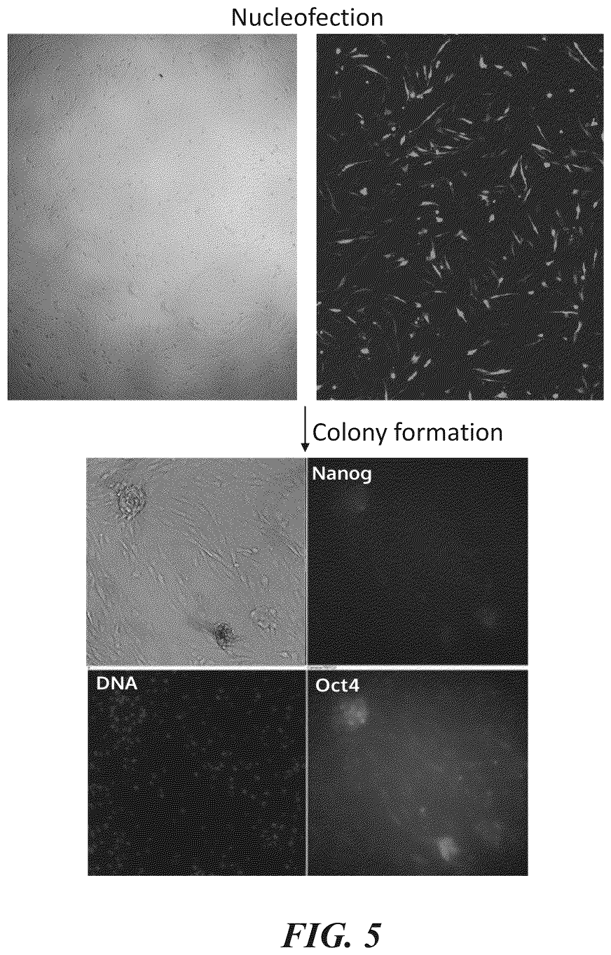

For transfection, each condition also included a gene encoding GFP to track transfection efficiency. As shown in FIG. 4 and top of FIG. 5, the transfection efficiency for the conditions was sufficient (e.g., greater than 80%). The three controls shown at the top of FIG. 4 from left to right correspond to conditions #1-3 of TABLE 1, respectively.

As shown in bottom of FIG. 5 and results compared in the right-most column of FIG. 3, HGC #6 yielded the highest rate of iPSC reprogramming, as ascertained by colony formation yield (e.g., after 5 days), as compared to other HGC conditions.

As shown in FIG. 3, sequentially activating the endogenous genes (e.g., condition 6) was more effective in iPSC reprogramming (e.g., 4 times more effective) than activating the endogenous genes substantially at once (e.g., condition 2).

As shown in FIG. 3, activating EEA sequentially (e.g., prior to) relative to activating one or more of the other genes (e.g., O, K, M, and/or S) (e.g., condition 6) was more effective in iPSC reprogramming than activating EEA and at least one of the other genes substantially at once (e.g., conditions 7, 8, and 9). Furthermore, even when EEA is activated sequentially (e.g., prior to) any of the other genes, sequentially activating the other genes (e.g., condition 6) was more effective in iPSC reprogramming than activating all of the other genes substantially at once (e.g., conditions 3-5).

As shown in FIG. 3, sequentially activating M and/or S twice (e.g., conditions 6, 7, and 8) was more effective in iPSC reprogramming than activating S only once (e.g., conditions 2, 3, 4, and 5).

### Example 3: Reprogramming of Primary Fibroblasts to iPSCs by Targeting Endogenous Genes (e.g., HERV)

### Targeting HERV and EEA

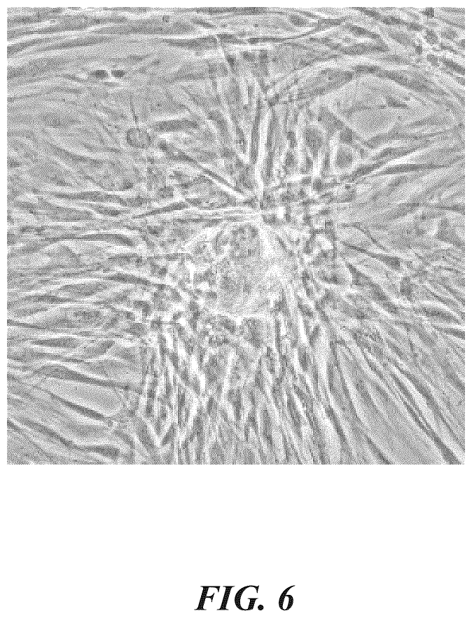

In accordance with Example 1 and similar to the procedures described in example 2, HGC condition #4 was utilized to target and modulate HERV (e.g., HERV-K) and EEA to reprogram primary fibroblasts to iPSCs. Upon transfection and subsequent culture (e.g., 5 days), targeting HERV and EEA, in the presence of anti-p53 shRNA, was sufficient to induce iPSC colony formation (FIG. 6).

### Targeting HERV

In accordance with Example 1 and similar to the procedures described in Example 2, HGCs can be designed to reprogram primary fibroblasts to iPSCs. A HGC can be designed for sequential activation of a plurality of genes. In some cases, a gene of the plurality of genes can be targeted via multiplex guide nucleic acid targeting (e.g., a single gate unit comprising a plurality of different gene regulating moiety proGuides for targeting different regions of the gene), while at least an additional of the plurality of genes can be targeted via single nucleic acid targeting (e.g., a single gate unit comprising a single gene regulating moiety proGuide for targeting a single region of the additional gene).

Briefly, primary fibroblasts were transfected with the respective HGC plasmids to modulate the following endogenous genes: Oct4 (O), Klf4 (K), c-Myc (M), Sox2 (S), and HERV-K element (e.g., LTR5HS; denoted herein as “H”).

TABLE 3 shows a library of different HGCs tested, each HGC designed to be capable of targeting one or more endogenous genes sequentially over four steps (step 1, step 2, step 3, and step 4). HGC #1-1 through 1-3 were designed as controls lacking modulation of HERV. HGC #2-1 through 2-4 were designed to test sequential targeting of the endogenous genes, including HERV, in various combinations and orders. Here, at each respective step of the HGC, KLF4 or MYC (e.g., KLF4 and MYC) were activated with a single guide nucleic acid targeting, while the other genes were activated via multiplex guide nucleic acid targeting.

Primary fibroblasts (e.g., about 500 k fibroblasts per well) were transfected with the respective HGC plasmids. In addition, the fibroblasts in each condition were also transfected with multiple plasmids, each encoding (i) Cas9-VPR, anti-p53 shRNA, miR-302, and GFP. After subsequent culture (e.g., 7 days), the cells were visually assessed to identify number of colonies exhibiting iPSC-like morphology, an indication of iPSC reprogramming.

As shown in the last column of TABLE 3, activation of HERV-K activation without activation of the other OSKM factors (e.g., condition 2-4) was able to generate iPSC morphology colonies.

As shown in the last column of TABLE 3, activation of HERV-K followed by delayed activation of the other OSKM factors (e.g., condition 2-2) was more effective in iPSC reprogramming than delayed activation of the other OSKM factors without prior HERV-K activation (e.g., condition 1-2).

As shown in the last column of TABLE 3, activation of HERV-K and O substantially at the same time (e.g., condition 2-3) was not as effective for iPSC reprogramming as comparable sequential activation of the OSKM factors in absence of HERV-K activation (e.g., condition 1-3). Similarly, activation of HERV-K and the other OSKM factors substantially at the same time (e.g., condition 2-1) was ineffective for iPSC reprogramming, similar to comparable activation of the OSKM factors in absence of HERV-K activation (e.g., condition 1-1).

Separately, in absence of HERV-K activation, sequentially activation the OSKM factors (e.g., condition 1-3) was more effective for iPSC reprogramming as compared to activating all of the OSKM factors substantially at the same time (e.g., conditions 1-1 and 1-2).

TABLE 3
Heterologous Genetic Circuits.

| | | | | | No. of iPSC |
|---|---|---|---|---|---|
| | | | | | morphology |
| Condition # | Step 1 | Step 2 | Step 3 | Step 4 | colonies |
| 1-1 | OSKM | — | — | — | 0 |
| 1-2 | — | OSKM | — | — | 2 |
| 1-3 | O | KM | S | S | 8 |
| 2-1 | H + OSKM | — | — | — | 0 |
| 2-2 | H | OSKM | — | — | 5 |
| 2-3 | H + O | KM | S | S | 0 |
| 2-4 | H | — | — | — | 2 |

### EMBODIMENTS

The following non-limiting embodiments provide illustrative examples of the invention, but do not limit the scope of the invention.

Embodiment 1. A method for conversion of a plurality of cells of a first cell type (first plurality of cells) into a plurality of cells of a second cell type (second plurality of cells), the method comprising:
contacting the first plurality of cells with a heterologous gene modulator exhibiting specific binding to a gene encoding HERV, to regulate an expression level or an epigenetic profile of HERV and effect the conversion from the first plurality of cells to the second plurality of cells,
optionally wherein:
(1) (i) the first plurality of cells comprises terminally differentiated cells and (ii) the second plurality of cells comprises pluripotent stem cells; and/or
(2) the contacting enhances the expression level of HERV; and/or
(3) the heterologous gene modulator exhibits specific binding to a gene encoding HERV-K;
and/or
(4) the heterologous gene modulator exhibits specific binding to a gene encoding LTR5HS, further optionally wherein:
   (a) the heterologous gene modulator exhibits specific binding to a polynucleotide sequence exhibiting at least about 80% sequence identity to the polypeptide sequence of one or more members selected from the group consisting of SEQ ID NOs: 1-12 and complementary sequences thereof, optionally with uracil-to-thymine conversion; and/or
   (b) the heterologous gene modulator exhibits specific binding to a polynucleotide sequence exhibiting at least about 90% sequence identity to the polypeptide sequence of one or more members selected from the group consisting of SEQ ID NOs: 1-12 and complementary sequences thereof, optionally with uracil-to-thymine conversion; and/or
   (c) the heterologous gene modulator comprises a polynucleotide sequence exhibiting at least about 80% sequence identity to the polypeptide sequence of one or more members selected from the group consisting of SEQ ID NOs: 1-12 and complementary sequences thereof, and/or
   (d) the heterologous gene modulator comprises a polynucleotide sequence exhibiting at least about 90% sequence identity to the polypeptide sequence of one or more members selected from the group consisting of SEQ ID NOs: 1-12 and complementary sequences thereof, and/or
(5) the contacting effects or is sufficient to effect the conversion without use of an additional heterologous gene modulator that exhibits specific binding to an additional target gene comprising one or more cell de-differentiation factors selected from the group consisting of OCT4, SOX2, KLF4, and MYC,
   further optionally wherein the additional target gene comprises OCT4, SOX2, KLF4, and MYC; and/or
(6) the method further comprises contacting the first plurality of cells with an additional heterologous gene modulator exhibiting specific binding to an additional gene comprising one or more cell de-differentiation factors selected from the group consisting of OCT4, SOX2, KLF4, and MYC, to effect a sequential regulation of the gene and the additional gene,
   further optionally wherein:
   (a) regulation of the gene occurs prior to, simultaneously with, or subsequent to regulation of the additional gene; and/or
   (b) the regulation of the gene occurs prior to the regulation of the additional gene; and/or
   (c) the additional gene comprises OCT4; and/or
(7) the method further comprises contacting the first plurality of cells with an additional heterologous gene modulator exhibiting specific binding to embryo genome activation (EGA)-enriched Alu-motif (EEA), to effect the conversion; and/or
(8) the contacting comprises contacting the first plurality of cells with a heterologous genetic circuit comprising a plurality of gate units, wherein the heterologous genetic circuit is activatable to induce the plurality of gate units to modulate expression level or epigenetic profile of the gene and a different gene in a sequential manner to effect the conversion, and wherein the plurality of gate units comprises:
   (i) a first gate unit comprising the heterologous gene modulator, wherein the first gate unit is preconfigured to regulate the expression level or epigenetic profile of the gene; and
   (ii) a second gate unit that is preconfigured to regulate expression level or epigenetic profile of the different gene,
   wherein, upon activation of the heterologous genetic circuit, the plurality of gate units operates to effect the conversion,
      further optionally wherein:
      (a) the heterologous genetic circuit is preconfigured to regulate the expression level of epigenetic profile of the gene prior to regulation of the expression level of epigenetic profile of the different gene; and/or
      (b) the different gene comprises one or more members selected from the group consisting of Oct3, Oct4, Sox2, Klf4, c-Myc, miR-302, miR-307, EEA, ZSCAN4, DUX4, OTX2, ABCE1, COL5A1, GAL4NT13, DUXA, DUXB, ARGFX, CPHX1, CPHX2, TPRX1, DPPA3, NASP, ATP2B1, NFAT, H1FOO, and CAMKII; and/or
      (c) the different gene comprises one or more members selected from the group consisting of Oct4, Sox2, Klf4, c-Myc, miR-302, miR-307, and EEA; and/or
(9) the heterologous gene modulator comprises a heterologous endonuclease for the specific binding to the gene encoding HERV,
   further optionally wherein:
   (a) the heterologous endonuclease is a Cas protein; and/or
   (b) the heterologous gene modulator further comprises a gene activator; and/or
   (c) the gene activator is selected from the group consisting of VP16, VP64, p65, RTA, and VP64-p65-RTA fusion (VPR); and/or
   (d) the gene activator is coupled to the heterologous endonuclease.

Embodiment 2. A system for conversion of a plurality of cells of a first cell type (first plurality of cells) into a plurality of cells of a second cell type (second plurality of cells), the system comprising:
a heterologous gene modulator exhibiting specific binding to a gene encoding HERV, to regulate an expression level or an epigenetic profile of the HERV and effect the conversion from the first plurality of cells to the second plurality of cells, optionally wherein:
(1) (i) the first plurality of cells comprises terminally differentiated cells and (ii) the second plurality of cells comprises pluripotent stem cells; and/or
(2) the specific binding of the heterologous gene modulator to the gene effect enhanced expression level of HERV; and/or
(3) the heterologous gene modulator exhibits specific binding to a gene encoding HERV-K;
and/or
(4) the heterologous gene modulator exhibits specific binding to a gene encoding LTR5HS,
   further optionally wherein:
   (a) the heterologous gene modulator exhibits specific binding to a polynucleotide sequence exhibiting at least about 80% sequence identity to the polypeptide sequence of one or more members selected from the group consisting of SEQ ID NOs: 1-12 and complementary sequences thereof, optionally with uracil-to-thymine conversion; and/or
   (b) the heterologous gene modulator exhibits specific binding to a polynucleotide sequence exhibiting at least about 90% sequence identity to the polypeptide sequence of one or more members selected from the group consisting of SEQ ID NOs: 1-12 and complementary sequences thereof, optionally with uracil-to-thymine conversion; and/or
   (c) the heterologous gene modulator comprises a polynucleotide sequence exhibiting at least about 80% sequence identity to the polypeptide sequence of one or more members selected from the group consisting of SEQ ID NOs: 1-12 and complementary sequences thereof, and/or
   (d) the heterologous gene modulator comprises a polynucleotide sequence exhibiting at least about 90% sequence identity to the polypeptide sequence of one or more members selected from the group consisting of SEQ ID NOs: 1-12 and complementary sequences thereof, and/or
(5) the specific binding of the heterologous gene modulator to the gene is configured to or is sufficient to effect the conversion without use of an additional heterologous gene modulator that exhibits specific binding to an additional target gene comprising one or more cell de-differentiation factors selected from the group consisting of OCT4, SOX2, KLF4, and MYC,
   further optionally wherein the additional target gene comprises OCT4, SOX2, KLF4, and MYC; and/or
(6) the system further comprises an additional heterologous gene modulator exhibiting specific binding to an additional gene comprising one or more cell de-differentiation factors selected from the group consisting of OCT4, SOX2, KLF4, and MYC, to effect a sequential regulation of the gene and the additional gene,
   further optionally wherein:
   (a) regulation of the gene occurs prior to, simultaneously with, or subsequent to regulation of the additional gene; and/or
   (b) the regulation of the gene occurs prior to the regulation of the additional gene; and/or
   (c) the additional gene comprises OCT4; and/or
(7) the system further comprises additional heterologous gene modulator exhibiting specific binding to embryo genome activation (EGA)-enriched Alu-motif (EEA), to effect the conversion;
and/or
(8) the system comprises a heterologous genetic circuit comprising a plurality of gate units, wherein the heterologous genetic circuit is activatable to induce the plurality of gate units to modulate expression level or epigenetic profile of the gene and a different gene in a sequential manner to effect the conversion, and wherein the plurality of gate units comprises:
   (i) a first gate unit comprising the heterologous gene modulator, wherein the first gate unit is preconfigured to regulate the expression level or epigenetic profile of the gene; and
   (ii) a second gate unit that is preconfigured to regulate expression level or epigenetic profile of the different gene,
   wherein, upon activation of the heterologous genetic circuit, the plurality of gate units operates to effect the conversion,
      further optionally wherein:
      (a) the heterologous genetic circuit is preconfigured to regulate the expression level of epigenetic profile of the gene prior to regulation of the expression level of epigenetic profile of the different gene; and/or
      (b) the different gene comprises one or more members selected from the group consisting of Oct3, Oct4, Sox2, Klf4, c-Myc, miR-302, miR-307, EEA, ZSCAN4, DUX4, OTX2, ABCE1, COL5A1, GAL4NT13, DUXA, DUXB, ARGFX, CPHX1, CPHX2, TPRX1, DPPA3, NASP, ATP2B1, NFAT, H1FOO, and CAMKII; and/or
      (c) the different gene comprises one or more members selected from the group consisting of Oct4, Sox2, Klf4, c-Myc, miR-302, miR-307, and EEA; and/or
(9) the heterologous gene modulator comprises a heterologous endonuclease for the specific binding to the gene encoding HERV,
   further optionally wherein:
   (a) the heterologous endonuclease is a Cas protein; and/or
   (b) the heterologous gene modulator further comprises a gene activator; and/or
   (c) the gene activator is selected from the group consisting of VP16, VP64, p65, RTA, and VP64-p65-RTA fusion (VPR); and/or
   (d) the gene activator is coupled to the heterologous endonuclease.

Embodiment 3. A method for conversion of a plurality of differentiated cells into a plurality of stem cells, the method comprising:
contacting the plurality of differentiated cells with a heterologous genetic circuit comprising a plurality of gate units, wherein the heterologous genetic circuit is activatable to induce the plurality of gate units to modulate expression level or epigenetic profile of a plurality of distinct target endogenous genes in a sequential manner to effect the conversion, and wherein the plurality of gate units comprises:
   (i) a first gate unit that is preconfigured to regulate expression level or epigenetic profile of a first target endogenous gene of the plurality of distinct target endogenous genes, wherein the first target endogenous gene comprises embryo genome activation (EGA)-enriched Alu-motif (EEA); and
   (ii) a second gate unit that is preconfigured to regulate expression level or epigenetic profile of a second target endogenous gene of the plurality of distinct target endogenous genes, wherein the second target endogenous gene comprises a cell de-differentiation factor selected from the group consisting of OCT4, SOX2, KLF4, and MYC,
wherein, upon activation of the heterologous genetic circuit, the plurality of gate units operates to effect the conversion,
optionally wherein:
(1) the plurality of stem cells comprises a plurality of pluripotent stem cells; and/or
(2) the heterologous genetic circuit is preconfigured such that, upon activation of the heterologous genetic circuit, (i) the expression level or epigenetic profile of the first target endogenous gene is regulated prior to (ii) the expression level or epigenetic profile of the second target endogenous gene; and/or
(3) the plurality of gate unit comprises a third gate unit that is preconfigured to regulate expression level or epigenetic profile of a third target endogenous gene of the plurality of distinct target endogenous genes, wherein the third target endogenous gene comprises a different cell de-differentiation factor selected from the group consisting of OCT4, SOX2, KLF4, and MYC,
   wherein, upon activation of the heterologous genetic circuit, expression levels or epigenetic profiles of the cell de-differentiation factor and the different cell de-differentiation factor are sequentially regulated,
      further optionally wherein:
      (a) the second target endogenous gene comprises OCT4; and/or
      (b) the third target endogenous gene comprises SOX2; and/or
(4) the plurality of gate unit comprises a third gate unit that is preconfigured to regulate expression level or epigenetic profile of the second target endogenous gene,
   wherein activation of the heterologous genetic circuit effects two or more sequential regulations of the expression level or epigenetic profile of the cell de-differentiation factor,
      further optionally wherein:
      (a) the cell de-differentiation factor is MYC; and/or
      (b) the cell de-differentiation factor is SOX2; and/or
(5) the cell de-differentiation factor comprises OCT4; and/or
(6) the cell de-differentiation factor comprises SOX2; and/or
(7) the cell de-differentiation factor comprises KLF4; and/or
(8) the cell de-differentiation factor comprises MYC.

Embodiment 4. A system for conversion of a plurality of differentiated cells into a plurality of stem cells, the system comprising:
a heterologous genetic circuit comprising a plurality of gate units, wherein the heterologous genetic circuit is activatable to induce the plurality of gate units to modulate expression level or epigenetic profile of a plurality of distinct target endogenous genes in a sequential manner to effect the conversion, wherein the plurality of gate units comprises:
   (i) a first gate unit that is preconfigured to regulate expression level or epigenetic profile of a first target endogenous gene of the plurality of distinct target endogenous genes, wherein the first target endogenous gene comprises embryo genome activation (EGA)-enriched Alu-motif (EEA); and
   (ii) a second gate unit that is preconfigured to regulate expression level or epigenetic profile of a second target endogenous gene of the plurality of distinct target endogenous genes, wherein the second target endogenous gene comprises a cell de-differentiation factor selected from the group consisting of OCT4, SOX2, KLF4, and MYC,
wherein, upon activation of the heterologous genetic circuit, the plurality of gate units operates to effect the conversion,
optionally wherein:
(1) the plurality of stem cells comprises a plurality of pluripotent stem cells; and/or
(2) the heterologous genetic circuit is preconfigured such that, upon activation of the heterologous genetic circuit, (i) the expression level or epigenetic profile of the first target endogenous gene is regulated prior to (ii) the expression level or epigenetic profile of the second target endogenous gene; and/or
(3) the plurality of gate unit comprises a third gate unit that is preconfigured to regulate expression level or epigenetic profile of a third target endogenous gene of the plurality of distinct target endogenous genes, wherein the third target endogenous gene comprises a different cell de-differentiation factor selected from the group consisting of OCT4, SOX2, KLF4, and MYC,
   wherein, upon activation of the heterologous genetic circuit, expression levels or epigenetic profiles of the cell de-differentiation factor and the different cell de-differentiation factor are sequentially regulated,
      further optionally wherein:
      (a) the second target endogenous gene comprises OCT4; and/or
      (b) the third target endogenous gene comprises SOX2; and/or
(4) the plurality of gate unit comprises a third gate unit that is preconfigured to regulate expression level or epigenetic profile of the second target endogenous gene,
   wherein activation of the heterologous genetic circuit effects two or more sequential regulations of the expression level or epigenetic profile of the cell de-differentiation factor,
      further optionally wherein:
      (a) the cell de-differentiation factor is MYC; and/or
      (b) the cell de-differentiation factor is SOX2; and/or
(5) the cell de-differentiation factor comprises OCT4; and/or
(6) the cell de-differentiation factor comprises SOX2; and/or
(7) the cell de-differentiation factor comprises KLF4; and/or
(8) the cell de-differentiation factor comprises MYC.

Embodiment 5. A method for conversion of a plurality of differentiated cells into a plurality of stem cells, the method comprising:
contacting the plurality of differentiated cells with a heterologous genetic circuit comprising a plurality of gate units, wherein the heterologous genetic circuit is activatable to induce the plurality of gate units to modulate expression level or epigenetic profile levels of a plurality of distinct target endogenous genes in a sequential manner to effect the conversion, and wherein the plurality of gate units comprises:
   (i) a first gate unit that is preconfigured to regulate expression level or epigenetic profile level of a first target endogenous gene of the plurality of distinct target endogenous genes, wherein the first target endogenous gene comprises a cell de-differentiation factor selected from the group consisting of OCT4, SOX2, KLF4, and MYC; and
   (ii) a second gate unit that is preconfigured to regulate expression level or epigenetic profile level of a second target endogenous gene of the plurality of distinct target endogenous genes, wherein the second target endogenous gene comprises a different cell de-differentiation factor selected from the group consisting of OCT4, SOX2, KLF4, and MYC,
wherein, upon activation of the heterologous genetic circuit, the plurality of gate units operates to effect the conversion,
optionally wherein:
(1) the heterologous genetic circuit is preconfigured such that activation of the first gate unit effects activation of the second gate unit, such that (i) regulation of the expression level or epigenetic profile level of the first target endogenous gene occurs prior to (ii) regulation of the expression level or epigenetic profile level of the second target endogenous gene,
   further optionally wherein the first target endogenous gene is OCT4, such that the regulation of the expression level or epigenetic profile level of OCT4 occurs prior to that of KLF4; and/or
(2) the plurality of stem cells comprises a plurality of pluripotent stem cells; and/or
(3) the first target endogenous gene comprises OCT4, and the second target endogenous gene comprises one or more members selected from the group consisting of SOX2, KLF4, and MYC; and/or
(4) the first target endogenous gene comprises one or more members selected from the group consisting of OCT4, KLF4, and MYC, and the second target endogenous gene comprises SOX2; and/or
(5) the plurality of gate unit further comprises a third gate unit that is preconfigured to regulate expression level or epigenetic profile level of the first target endogenous gene or the second target endogenous gene, to effect two or more sequential regulations of the first target endogenous gene or the second target endogenous gene, respectively,
   further optionally wherein SOX2 is subjected to the two or more sequential regulations; and/or
(6) modulation of the expression level or epigenetic profile levels of the plurality of distinct target endogenous genes comprises enhancing the expression level or epigenetic profile levels of the plurality of distinct target endogenous genes.

Embodiment 6. A system for conversion of a plurality of differentiated cells into a plurality of stem cells, the system comprising:
a heterologous genetic circuit comprising a plurality of gate units, wherein the heterologous genetic circuit is activatable to induce the plurality of gate units to modulate expression level or epigenetic profile levels of a plurality of distinct target endogenous genes in a sequential manner to effect the conversion, and wherein the plurality of gate units comprises:
   (i) a first gate unit that is preconfigured to regulate expression level or epigenetic profile level of a first target endogenous gene of the plurality of distinct target endogenous genes, wherein the first target endogenous gene comprises a cell de-differentiation factor selected from the group consisting of OCT4, SOX2, KLF4, and MYC; and
   (ii) a second gate unit that is preconfigured to regulate expression level or epigenetic profile level of a second target endogenous gene of the plurality of distinct target endogenous genes, wherein the second target endogenous gene comprises a different cell de-differentiation factor selected from the group consisting of OCT4, SOX2, KLF4, and MYC,
wherein, upon activation of the heterologous genetic circuit, the plurality of gate units operates to effect the conversion,
optionally wherein:
(1) the heterologous genetic circuit is preconfigured such that activation of the first gate unit effects activation of the second gate unit, such that (i) regulation of the expression level or epigenetic profile level of the first target endogenous gene occurs prior to (ii) regulation of the expression level or epigenetic profile level of the second target endogenous gene,
   further optionally wherein the first target endogenous gene is OCT4, such that the regulation of the expression level or epigenetic profile level of OCT4 occurs prior to that of KLF4; and/or
(2) the plurality of stem cells comprises a plurality of pluripotent stem cells; and/or
(3) the first target endogenous gene comprises OCT4, and the second target endogenous gene comprises one or more members selected from the group consisting of SOX2, KLF4, and MYC; and/or
(4) the first target endogenous gene comprises one or more members selected from the group consisting of OCT4, KLF4, and MYC, and the second target endogenous gene comprises SOX2; and/or
(5) the plurality of gate unit further comprises a third gate unit that is preconfigured to regulate expression level or epigenetic profile level of the first target endogenous gene or the second target endogenous gene, to effect two or more sequential regulations of the first target endogenous gene or the second target endogenous gene, respectively,
   further optionally wherein:
   (a) SOX2 is subjected to the two or more sequential regulations; and/or
   (b) modulation of the expression level or epigenetic profile levels of the plurality of distinct target endogenous genes comprises enhancing the expression level or epigenetic profile levels of the plurality of distinct target endogenous genes.

Additional details of heterologous genetic circuits (HGC) and uses thereof are provided in International Application No. PCT/US2018/052211 (entitled “CRISPR/CAS SYSTEM AND METHOD FOR GENOME EDITING AND MODULATING TRANSCRIPTION”), International Application No. PCT/US2023/013240 (entitled “SYSTEMS FOR CELL PROGRAMMING AND METHODS THEREOF), and Clarke et al., Molecular Cell, 81, 226-238, 2021 (entitled “Sequential Activation of Guide RNAs to Enable Successive CRISPR-Cas9 Activities”), each of which is incorporated herein by reference in its entirety.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-70. (canceled)

71. A method for conversion of a plurality of cells of a first cell type (first plurality of cells) into a plurality of cells of a second cell type (second plurality of cells), the method comprising:
contacting the first plurality of cells with a heterologous gene modulator exhibiting specific binding to a gene encoding HERV, to regulate an expression level or an epigenetic profile of HERV and effect the conversion from the first plurality of cells to the second plurality of cells.

72. The method of claim 71, wherein (i) the first plurality of cells comprises terminally differentiated cells and (ii) the second plurality of cells comprises pluripotent stem cells.

73. The method of claim 71, wherein the contacting enhances the expression level of HERV.

74. The method of claim 71, wherein the heterologous gene modulator exhibits specific binding to a gene encoding HERV-K.

75. The method of claim 71, wherein the heterologous gene modulator exhibits specific binding to a gene encoding LTR5HS.

76. The method of claim 75, wherein the heterologous gene modulator exhibits specific binding to a polynucleotide sequence exhibiting at least about 80% sequence identity to the polypeptide sequence of one or more members selected from the group consisting of SEQ ID NOs: 1-12 and complementary sequences thereof, optionally with uracil-to-thymine conversion.

77. The method of claim 75, wherein the heterologous gene modulator comprises a polynucleotide sequence exhibiting at least about 80% sequence identity to the polypeptide sequence of one or more members selected from the group consisting of SEQ ID NOs: 1-12 and complementary sequences thereof.

78. The method of claim 71, wherein the contacting effects or is sufficient to effect the conversion without use of an additional heterologous gene modulator that exhibits specific binding to an additional target gene comprising one or more cell de-differentiation factors selected from the group consisting of OCT4, SOX2, KLF4, and MYC.

79. The method of claim 71, further comprising contacting the first plurality of cells with an additional heterologous gene modulator exhibiting specific binding to an additional gene comprising one or more cell de-differentiation factors selected from the group consisting of OCT4, SOX2, KLF4, and MYC, to effect a sequential regulation of the gene and the additional gene.

80. The method of claim 79, wherein regulation of the gene occurs prior to, simultaneously with, or subsequent to regulation of the additional gene.

81. The method of claim 80, wherein the additional gene comprises OCT4.

82. The method of claim 71, further comprising contacting the first plurality of cells with an additional heterologous gene modulator exhibiting specific binding to embryo genome activation (EGA)-enriched Alu-motif (EEA), to effect the conversion.

83. The method of claim 71, wherein the contacting comprises contacting the first plurality of cells with a heterologous genetic circuit comprising a plurality of gate units, wherein the heterologous genetic circuit is activatable to induce the plurality of gate units to modulate expression level or epigenetic profile of the gene and a different gene in a sequential manner to effect the conversion, and wherein the plurality of gate units comprises:
(i) a first gate unit comprising the heterologous gene modulator, wherein the first gate unit is preconfigured to regulate the expression level or epigenetic profile of the gene; and
(ii) a second gate unit that is preconfigured to regulate expression level or epigenetic profile of the different gene,
wherein, upon activation of the heterologous genetic circuit, the plurality of gate units operates to effect the conversion.

84. The method of claim 83, wherein the activation of the heterologous genetic circuit is a single activation.

85. The method of claim 83, wherein the heterologous genetic circuit is preconfigured to regulate the expression level of epigenetic profile of the gene prior to regulation of the expression level of epigenetic profile of the different gene.

86. The method of claim 83, wherein the different gene comprises one or more members selected from the group consisting of Oct3, Oct4, Sox2, Klf4, c-Myc, miR-302, miR-307, EEA, ZSCAN4, DUX4, OTX2, ABCE1, COL5A1, GAL4NT13, DUXA, DUXB, ARGFX, CPHX1, CPHX2, TPRX1, DPPA3, NASP, ATP2B1, NFAT, H1FOO, and CAMKII.

87. The method of claim 71, wherein the heterologous gene modulator comprises a heterologous endonuclease for the specific binding to the gene encoding HERV.

88. The method of claim 87, wherein the heterologous gene modulator further comprises a gene activator.

89. A system for conversion of a plurality of cells of a first cell type (first plurality of cells) into a plurality of cells of a second cell type (second plurality of cells), the system comprising:
a heterologous gene modulator exhibiting specific binding to a gene encoding HERV, to regulate an expression level or an epigenetic profile of the HERV and effect the conversion from the first plurality of cells to the second plurality of cells.

90. A method for treating a subject in need thereof, the method comprising administering to the subject a plurality of cells generated via contacting a plurality of cells of a first cell type (first plurality of cells) with a heterologous gene modulator to effect conversion of the first plurality of cells into a plurality of cells of a second cell type (second plurality of cells),
wherein the heterologous gene modulator exhibits specific binding to a gene encoding HERV, to regulate an expression level or an epigenetic profile of the HERV and effect the conversion from the first plurality of cells to the second plurality of cells.


SEQUENCE LISTING (63 sequences)

SEQ ID NO: 1 - RNA, 16 nt, synthetic construct
  source 1..16: mol_type = other RNA
tactaagggaactcag

SEQ ID NO: 2 - RNA, 16 nt, synthetic construct
  source 1..16: mol_type = other RNA
aaaaccgccttagggc

SEQ ID NO: 3 - RNA, 16 nt, synthetic construct
  source 1..16: mol_type = other RNA
aaaactgccttagggc

SEQ ID NO: 4 - RNA, 16 nt, synthetic construct
  source 1..16: mol_type = other RNA
agcccgacacccgtaa

SEQ ID NO: 5 - RNA, 16 nt, synthetic construct
  source 1..16: mol_type = other RNA
aggagaaaaccacctt

SEQ ID NO: 6 - RNA, 16 nt, synthetic construct
  source 1..16: mol_type = other RNA
taccatctcggagagg

SEQ ID NO: 7 - RNA, 16 nt, synthetic construct
  source 1..16: mol_type = other RNA
aaccttggacaatacc

SEQ ID NO: 8 - RNA, 16 nt, synthetic construct
  source 1..16: mol_type = other RNA
taaagggtctgtgctg

SEQ ID NO: 9 - RNA, 16 nt, synthetic construct
  source 1..16: mol_type = other RNA
gacattccattgccca

SEQ ID NO: 10 - RNA, 16 nt, synthetic construct
  source 1..16: mol_type = other RNA
atagagaaagaaaagt

SEQ ID NO: 11 - RNA, 16 nt, synthetic construct
  source 1..16: mol_type = other RNA
gaaacgcccacaggtg

SEQ ID NO: 12 - RNA, 16 nt, synthetic construct
  source 1..16: mol_type = other RNA
aagctttcttgcttag

SEQ ID NO: 13 - RNA, 14 nt, synthetic construct
  source 1..14: mol_type = other RNA
actggcgcccctcc

SEQ ID NO: 14 - RNA, 14 nt, synthetic construct
  source 1..14: mol_type = other RNA
agaaactgaggcga

SEQ ID NO: 15 - RNA, 14 nt, synthetic construct
  source 1..14: mol_type = other RNA
ggcaatggtgtctg

SEQ ID NO: 16 - RNA, 14 nt, synthetic construct
  source 1..14: mol_type = other RNA
ggggacctgcactg

SEQ ID NO: 17 - RNA, 14 nt, synthetic construct
  source 1..14: mol_type = other RNA
gtgccagaggtctg

SEQ ID NO: 18 - RNA, 20 nt, synthetic construct
  source 1..20: mol_type = other RNA
aagaatagtataaatagaag

SEQ ID NO: 19 - RNA, 20 nt, synthetic construct
  source 1..20: mol_type = other RNA
atctcagagaatcattacaa

SEQ ID NO: 20 - RNA, 20 nt, synthetic construct
  source 1..20: mol_type = other RNA
agggaatgtatgatcctggg

SEQ ID NO: 21 - RNA, 20 nt, synthetic construct
  source 1..20: mol_type = other RNA
aaaaggatccagacccaccc

SEQ ID NO: 22 - RNA, 20 nt, synthetic construct
  source 1..20: mol_type = other RNA
tttaagaggaagatatcttg

SEQ ID NO: 23 - RNA, 20 nt, synthetic construct
  source 1..20: mol_type = other RNA
atgccatcaaacaagcagat

SEQ ID NO: 24 - RNA, 20 nt, synthetic construct
  source 1..20: mol_type = other RNA
caatgcctttctcggctcag

SEQ ID NO: 25 - RNA, 14 nt, synthetic construct
  source 1..14: mol_type = other RNA
taaggaacgcgcgc

SEQ ID NO: 26 - RNA, 14 nt, synthetic construct
  source 1..14: mol_type = other RNA
tgtctgcgggcgcg

SEQ ID NO: 27 - RNA, 14 nt, synthetic construct
  source 1..14: mol_type = other RNA
cgggcttcgaaccc

SEQ ID NO: 28 - RNA, 14 nt, synthetic construct
  source 1..14: mol_type = other RNA
atagcaacgatgga

SEQ ID NO: 29 - RNA, 14 nt, synthetic construct
  source 1..14: mol_type = other RNA
tcgctgcgcgacca

SEQ ID NO: 30 - RNA, 14 nt, synthetic construct
  source 1..14: mol_type = other RNA
ggggcacacaaagc

SEQ ID NO: 31 - RNA, 14 nt, synthetic construct
  source 1..14: mol_type = other RNA
cctgttccagagct

SEQ ID NO: 32 - RNA, 14 nt, synthetic construct
  source 1..14: mol_type = other RNA
caaaccggcgccac

SEQ ID NO: 33 - RNA, 14 nt, synthetic construct
  source 1..14: mol_type = other RNA
ggtacccgaaccgc

SEQ ID NO: 34 - RNA, 14 nt, synthetic construct
  source 1..14: mol_type = other RNA
cggggcaggagggg

SEQ ID NO: 35 - RNA, 14 nt, synthetic construct
  source 1..14: mol_type = other RNA
caaagcagagggcg

SEQ ID NO: 36 - RNA, 14 nt, synthetic construct
  source 1..14: mol_type = other RNA
ggtggggtacagac

SEQ ID NO: 37 - RNA, 14 nt, synthetic construct
  source 1..14: mol_type = other RNA
acccagccgcttta

SEQ ID NO: 38 - RNA, 14 nt, synthetic construct
  source 1..14: mol_type = other RNA
tggcgcagacaagg

SEQ ID NO: 39 - RNA, 14 nt, synthetic construct
  source 1..14: mol_type = other RNA
cagggcacgggcgc

SEQ ID NO: 40 - RNA, 14 nt, synthetic construct
  source 1..14: mol_type = other RNA
cgcagacaaggggg

SEQ ID NO: 41 - RNA, 14 nt, synthetic construct
  source 1..14: mol_type = other RNA
taatctccgcccac

SEQ ID NO: 42 - RNA, 14 nt, synthetic construct
  source 1..14: mol_type = other RNA
gtaagagaggagag

SEQ ID NO: 43 - RNA, 14 nt, synthetic construct
  source 1..14: mol_type = other RNA
gccgccggcgggcc

SEQ ID NO: 44 - RNA, 14 nt, synthetic construct
  source 1..14: mol_type = other RNA
gcgggggaggccgg

SEQ ID NO: 45 - RNA, 14 nt, synthetic construct
  source 1..14: mol_type = other RNA
cccaaagtcccggc

SEQ ID NO: 46 - RNA, 14 nt, synthetic construct
  source 1..14: mol_type = other RNA
cactaagactacgt

SEQ ID NO: 47 - RNA, 14 nt, synthetic construct
  source 1..14: mol_type = other RNA
cgcagggctccgcg

SEQ ID NO: 48 - RNA, 14 nt, synthetic construct
  source 1..14: mol_type = other RNA
ggcaggctggctct

SEQ ID NO: 49 - RNA, 14 nt, synthetic construct
  source 1..14: mol_type = other RNA
aaggtgcgggggtg

SEQ ID NO: 50 - RNA, 14 nt, synthetic construct
  source 1..14: mol_type = other RNA
gcgggcggggggag

SEQ ID NO: 51 - RNA, 14 nt, synthetic construct
  source 1..14: mol_type = other RNA
cacttccttcgaaa

SEQ ID NO: 52 - RNA, 14 nt, synthetic construct
  source 1..14: mol_type = other RNA
cgccgcgcgctgat

SEQ ID NO: 53 - RNA, 14 nt, synthetic construct
  source 1..14: mol_type = other RNA
tggcaggctggctc

SEQ ID NO: 54 - RNA, 14 nt, synthetic construct
  source 1..14: mol_type = other RNA
tcatcggcggcggc

SEQ ID NO: 55 - RNA, 14 nt, synthetic construct
  source 1..14: mol_type = other RNA
gaccaatcagcgcg

SEQ ID NO: 56 - RNA, 14 nt, synthetic construct
  source 1..14: mol_type = other RNA
agtgtttgcaaaag

SEQ ID NO: 57 - RNA, 14 nt, synthetic construct
  source 1..14: mol_type = other RNA
catgaaagggggcg

SEQ ID NO: 58 - RNA, 14 nt, synthetic construct
  source 1..14: mol_type = other RNA
ccggcagcgaggct

SEQ ID NO: 59 - RNA, 14 nt, synthetic construct
  source 1..14: mol_type = other RNA
aatcccagcacttt

SEQ ID NO: 60 - RNA, 14 nt, synthetic construct
  source 1..14: mol_type = other RNA
gctacttgggaggc

SEQ ID NO: 61 - RNA, 14 nt, synthetic construct
  source 1..14: mol_type = other RNA
gcctcccaagtagc

SEQ ID NO: 62 - RNA, 14 nt, synthetic construct
  source 1..14: mol_type = other RNA
gcctcccaaagtgc

SEQ ID NO: 63 - RNA, 14 nt, synthetic construct
  source 1..14: mol_type = other RNA
cccagcactttggg